(19) United States
(12) Patent Application Publication
(10) Pub. No.: US 2025/0066499 A1
(43) Pub. Date: Feb. 27, 2025

(54) ANTIBODY BINDING TO HUMAN CD73, PREPARATION METHOD THEREFOR, AND USE THEREOF

(71) Applicant: SUNSHINE GUOJIAN PHARMACEUTICAL (SHANGHAI) CO., LTD., Pudong New Area, Shanghai (CN)

(72) Inventors: Xuesai Zhang, Pudong New Area, Shanghai (CN); Haomin Huang, Pudong New Area, Shanghai (CN); Zhenping Zhu, Pudong New Area, Shanghai (CN); Shaorong Deng, Pudong New Area, Shanghai (CN); Jianhe Chen, Pudong New Area, Shanghai (CN)

(73) Assignee: SUNSHINE GUOJIAN PHARMACEUTICAL (SHANGHAI) CO., LTD., Pudong New Area, Shanghai (CN)

(21) Appl. No.: 18/720,951

(22) PCT Filed: Dec. 16, 2022
(86) PCT No.: PCT/CN2022/139741

§ 371 Date: Jun. 17, 2024

(30) Foreign Application Priority Data
Dec. 17, 2021 (CN) 202111552957.3

(51) Int. Cl. C07K 16/28

(52) U.S. Cl. CPC C07K 16/2896; C07K 2317/24; C07K 2317/33; C07K 2317/76; C07K 2317/92

(57) ABSTRACT

The present invention provides an antibody binding to human CD73, a preparation method therefor, and a use thereof. The monoclonal antibody of the present invention can bind to a CD73 antigen with high specificity, has high affinity, has remarkable anti-tumor activity and the like, and has excellent clinical application prospects.

### TECHNICAL FIELD

The present invention belongs to the field of tumor therapy. Specifically, it relates to an antibody binding to human CD73, a preparation method therefor, and a use thereof.

### BACKGROUND

Currently, with the progress of researches on PD-1/PD-L1 and CTLA-4 and the like in the field of tumor immunotherapy, immunotherapy has become one of the main directions for conquering cancers. However, the existing tumor immunotherapy methods have a low efficiency rate and still cannot meet the clinical needs. Studies have found that one of the main reasons for the poor response of tumor immunotherapy is the presence of substances inhibiting immune cells in the tumor microenvironment, which leads to the escape of tumor cells from the killing of immune cells.

Adenosine is one of the important substances that produce tumor immunosuppression in the tumor microenvironment. By binding to adenosine receptors (A2AR), it activates protein kinase A (PKA) and Csk kinase, inhibits a series of signal pathways related to immune activation such as LCK, MAPK, PKC, and exerts an immunosuppressive effect. On the one hand, high concentrations of adenosine impair the activation and function of T cells and natural killer (NK) cells, leading to strong immunosuppression; on the other hand, high concentrations of adenosine enhance the function of regulatory T cells (Treg) and the differentiation of macrophage M2. In the process of adenosine production, there are two important key links: 1) when there is tissue disorder in the body (such as inflammation, malignant tumors, etc.), intracellular ATPs will be released into the extracellular space in large quantities, and these ATPs will be hydrolyzed by extracellular nucleotide hydrolase CD39 into ADPs and AMPs; 2) AMPs are then dephosphorylated to generate immunosuppressive adenosines under the synergistic action of CD73.

CD73 is an extracellular-5′-nucleotidase encoded by the NT5E gene. It has a molecular weight of 70 kD and is one of the main rate-limiting enzymes for adenosine production in the body. The expression of CD73 is regulated by molecules such as hypoxia-inducible factor-1 (HIF-1), TGF-β, EGFR, AKT, and β-catenin, wherein HIF-1, which functions as a transcription factor, is the most critical. Hypoxia is an important feature of the tumor microenvironment, which induces the upregulation of HIF-1 in the tumor microenvironment, leading to widespread expression of CD73 in tumors. Therefore, studies have found that CD73 is overexpressed on the surface of various tumors and is closely related to poor prognosis of tumors, including breast cancer, lung cancer, ovarian cancer, colorectal cancer, renal cancer, gastric cancer, head and neck cancer, etc.

Preclinical studies have shown that inhibiting CD73 can stimulate the activity of T cells and enhance anti-tumor immune surveillance on the levels of T cells and other immune cells regulated by adenosines. Relieving the inhibitory effect of the tumor microenvironment (TME) on immune effector cells is an important aspect of overcoming immunotherapy resistance and improving efficacy.

However, there are still many shortcomings in the numerous anti-CD73 antibodies in this field. Therefore, there is a need in this field to develop anti-CD73 antibodies suitable for treating patients.

### SUMMARY OF THE INVENTION

The purpose of the present invention is to provide an antibody binding to human CD73, a preparation method therefor, and a use thereof.

In the first aspect of the present invention, it provides an anti-human CD73 antibody or an antigen-binding fragment thereof, wherein the antibody or the antigen-binding fragment thereof comprises a heavy chain variable region and a light chain variable region, wherein,
the heavy chain variable region comprises three heavy chain complementarity determining regions (CDRs):
HCDR1 represented by SEQ ID NO. 10,
HCDR2 represented by SEQ ID NO. 11,
HCDR3 represented by SEQ ID NO. 12; or
HCDR1 represented by SEQ ID NO. 16,
HCDR2 represented by SEQ ID NO. 17,
HCDR3 represented by SEQ ID NO. 18; and
the light chain variable region comprises three light chain complementarity determining regions (CDRs):
LCDR1 represented by SEQ ID NO. 13,
LCDR2 represented by SEQ ID NO. 14,
LCDR3 represented by SEQ ID NO. 15; or
LCDR1 represented by SEQ ID NO. 19,
LCDR2 represented by SEQ ID NO. 20,
LCDR3 represented by SEQ ID NO. 21.

Any one of the amino acid sequences of the antibody or the antigen-binding fragment thereof further comprises a derivative sequence that is optionally with at least one amino acid added, deleted, modified, and/or substituted, and is capable of retaining the affinity for binding to CD73.

In another preferred embodiment, the amino acid sequence of any one of the CDRs mentioned above comprises a derivative CDR sequence that is with 1, 2, or 3 amino acids added, deleted, modified, and/or substituted, and allows the derivative antibody consisting of the VH and VL comprising the derivative CDR sequence to be capable of retaining the affinity for binding to CD73.

In another preferred embodiment, the number of added, deleted, modified and/or substituted amino acids is 1-5 (such as 1-3, preferably 1-2, and more preferably 1).

In another preferred embodiment, the antibody comprises a heavy chain and a light chain, wherein the heavy chain of the antibody comprises the three heavy chain complementarity determining regions (CDRs) and heavy chain framework regions for connecting the heavy chain complementarity determining regions (CDRs); and the light chain of the antibody comprises the three light chain complementarity determining regions (CDRs) and light chain framework regions for connecting the light chain complementarity determining regions (CDRs).

In another preferred embodiment, the heavy chain variable region has an amino acid sequence represented by SEQ ID NO. 1, 4, 6, 9, 24, or 28; preferably, it has an amino acid sequence represented by SEQ ID NO. 1, 4, 6, or 9.

In another preferred embodiment, the heavy chain further comprises a heavy chain constant region.

In another preferred embodiment, the heavy chain constant region is of human origin or murine origin.

In another preferred embodiment, the heavy chain constant region is a human antibody heavy chain IgG1 or IgG4 constant region.

In another preferred embodiment, the sequence of the heavy chain constant region is represented by SEQ ID NO. 31.

In another preferred embodiment, the light chain variable region has an amino acid sequence represented by SEQ ID NO. 2, 3, 5, 7, 8, 26, or 30; preferably, it has an amino acid sequence represented by SEQ ID NO. 2, 3, 5, 7, or 8.

In another preferred embodiment, the light chain further comprises a light chain constant region.

In another preferred embodiment, the light chain constant region is of human origin or murine origin.

In another preferred embodiment, the light chain constant region is a human antibody light chain kappa or lambda constant region.

In another preferred embodiment, the sequence of the light chain constant region is represented by SEQ ID NO. 32.

In another preferred embodiment, the antibody further comprises a heavy chain constant region and/or a light chain constant region.

In another preferred embodiment, the heavy chain constant region is of human origin, and/or the light chain constant region is of human origin.

In another preferred embodiment, the heavy chain constant region is a human antibody heavy chain IgG4 (S228P) constant region, and the light chain constant region is a human antibody light chain kappa constant region.

In another preferred embodiment, the heavy chain variable region of the antibody further comprises a human-derived framework region, and/or the light chain variable region of the antibody further comprises a human-derived framework region.

In another preferred embodiment, the heavy chain variable region of the antibody further comprises a murine-derived framework region, and/or the light chain variable region of the antibody further comprises a murine-derived framework region.

In another preferred embodiment, the antibody is selected from the group consisting of: an animal-derived antibody, a chimeric antibody, a humanized antibody, a fully human antibody, and a combination thereof.

In another preferred embodiment, the antibody is a partially or fully humanized, or fully human monoclonal antibody.

In another preferred embodiment, the antibody is a double-chain antibody or a single-chain antibody.

In another preferred embodiment, the antibody is a full-length antibody protein or an antigen-binding fragment.

In another preferred embodiment, the antigen-binding fragment comprises a Fab fragment, a F(ab′)₂ fragment, or a Fv fragment.

In another preferred embodiment, the antibody is a bispecific antibody or a multi-specific antibody.

In another preferred embodiment, the antibody is in the form of a drug conjugate.

In another preferred embodiment, the antibody or the antigen-binding fragment thereof comprises a heavy chain variable region and a light chain variable region; wherein, the heavy chain variable region comprises the following three complementarity determining regions (CDRs):
HCDR1 represented by SEQ ID NO. 10,
HCDR2 represented by SEQ ID NO. 11,
HCDR3 represented by SEQ ID NO. 12; and
the light chain variable region comprises the following three complementarity determining regions (CDRs):
LCDR1 represented by SEQ ID NO. 13,
LCDR2 represented by SEQ ID NO. 14,
LCDR3 represented by SEQ ID NO. 15; or
the heavy chain variable region comprises the following three complementarity determining regions (CDRs):
HCDR1 represented by SEQ ID NO. 16,
HCDR2 represented by SEQ ID NO. 17,
HCDR3 represented by SEQ ID NO. 18; and
the light chain variable region comprises the following three complementarity determining regions (CDRs):
LCDR1 represented by SEQ ID NO. 19,
LCDR2 represented by SEQ ID NO. 20,
LCDR3 represented by SEQ ID NO. 21.

In another preferred embodiment, the heavy chain variable region of the antibody comprises any one of the amino acid sequences represented by 1, 4, 6, 9, 24, or 28; and/or the light chain variable region comprises any one of the amino acid sequences represented by 2, 3, 5, 7, 8, 26, or 30.

In another preferred embodiment, the heavy chain variable region of the antibody comprises the amino acid sequence represented by SEQ ID NO. 1, and the light chain variable region of the antibody comprises the amino acid sequence represented by SEQ ID NO. 2; or the heavy chain variable region comprises the amino acid sequence represented by SEQ ID NO. 1, and the light chain variable region comprises the amino acid sequence represented by SEQ ID NO. 3; or the heavy chain variable region comprises the amino acid sequence represented by SEQ ID NO. 4, and the light chain variable region comprises the amino acid sequence represented by SEQ ID NO. 2; or the heavy chain variable region comprises the amino acid sequence represented by SEQ ID NO. 4, and the light chain variable region comprises the amino acid sequence represented by SEQ ID NO. 5; or the heavy chain variable region comprises the amino acid sequence represented by SEQ ID NO. 4, and the light chain variable region comprises the amino acid sequence represented by SEQ ID NO. 3; or the heavy chain variable region comprises the amino acid sequence represented by SEQ ID NO. 6, and the light chain variable region comprises the amino acid sequence represented by SEQ ID NO. 7; or the heavy chain variable region comprises the amino acid sequence represented by SEQ ID NO. 6, and the light chain variable region comprises the amino acid sequence represented by SEQ ID NO. 8; or the heavy chain variable region comprises the amino acid sequence represented by SEQ ID NO. 9, and the light chain variable region comprises the amino acid sequence represented by SEQ ID NO. 8.

In another preferred embodiment, the amino acid sequence of the heavy chain variable region has at least 80%, 85%, 90%, 91%, 92%, 93%, 94%, 95%, 96%, 97%, 98%, or 99% sequence homology or sequence identity to the amino acid sequence represented by SEQ ID NO. 1, 4, 6, 9, 24, or 28.

In another preferred embodiment, the amino acid sequence of the light chain variable region has at least 80%, 85%, 90%, 91%, 92%, 93%, 94%, 95%, 96%, 97%, 98%, or 99% sequence homology or sequence identity to the amino acid sequence represented by SEQ ID NO. 2, 3, 5, 7, 8, 26, or 30.

In another preferred embodiment, the antibody is a humanized antibody, the heavy chain variable region (VH) and light chain variable region (VL) of the antibody comprise amino acid sequences selected from Table 3.

In another preferred embodiment, the binding epitope of the antibody to human CD73 protein comprises a site corresponding to the CD73 extracellular domain (SEQ ID NO. 22) selected from the group consisting of:
tyrosine at position 132 (Y132), leucine at position 133 (L133), proline at position 139 (P139), valine at position 137 (V137), leucine at position 181 (L181), leucine at position 184 (L184), valine at position 144 (V144), lysine at position 180 (K180).

In the second aspect of the present invention, it provides a recombinant protein, which comprises:
(i) the antibody or the antigen-binding fragment thereof of the first aspect of the present invention; and
(ii) optionally a tag sequence for assisting expression and/or purification.

In another preferred embodiment, the tag sequence comprises a 6×His tag.

In another preferred embodiment, the recombinant protein (or polypeptide) comprises a fusion protein.

In another preferred embodiment, the recombinant protein is a monomer, a dimer, or a polymer.

In another preferred embodiment, the recombinant protein comprises:
(i) an antibody selected from the group consisting of: the heavy chain variable region of the antibody comprises any one of the amino acid sequences represented by 1, 4, 6, 9, 24, or 28; and/or the light chain variable region comprises any one of the amino acid sequences represented by 2, 3, 5, 7, 8, 26, or 30; and (ii) optionally a tag sequence for assisting expression and/or purification.

In another preferred embodiment, the recombinant protein further comprises an antibody or an antigen-binding fragment thereof that binds to other targets, such as an antibody or an antigen-binding fragment thereof that binds to CTLA-4, PD-1, or PD-L1.

In the third aspect of the present invention, it provides a polynucleotide, which encodes a polypeptide selected from the group consisting of:
(1) the antibody or the antigen-binding fragment thereof of the first aspect of the present invention; and
(2) the recombinant protein of the second aspect of the present invention.

In another preferred embodiment, the polynucleotide encoding the heavy chain variable region is represented by SEQ ID NO. 23, 27, 33, 36, 38, 41; and/or, the polynucleotide encoding the light chain variable region is represented by SEQ ID NO. 25, 29, 34, 35, 37, 39, 40.

In the fourth aspect of the present invention, it provides a vector, which comprises the polynucleotide of the third aspect of the present invention.

In another preferred embodiment, the vector comprises: a bacterial plasmid, a bacteriophage, a yeast plasmid, a plant cell virus, a mammalian cell virus such as an adenovirus, a retrovirus, or other vectors.

In the fifth aspect of the present invention, it provides a genetically engineered host cell, which comprises the vector of the fourth aspect of the present invention or has the polynucleotide of the third aspect of the present invention integrated into its genome.

In the sixth aspect of the present invention, it provides an antibody conjugate, which comprises:
(a) an antibody moiety, which is the antibody or the antigen-binding fragment thereof of the first aspect of the present invention; and
(b) a conjugate moiety coupled to the antibody moiety, which is selected from the group consisting of: a detectable label, a drug, a toxin, a cytokine, a radionuclide, an enzyme, and a combination thereof.

In another preferred embodiment, the antibody moiety is coupled to the conjugate moiety through a chemical bond or a linker.

In the seventh aspect of the present invention, it provides a CAR construct, wherein the scFv segment of the monoclonal antibody antigen-binding region of the CAR construct is a binding region that specifically binds to CD73, and the heavy chain variable region of the scFv comprises:
wherein the heavy chain variable region of the scFv comprises the following three complementarity determining regions (CDRs):
HCDR1 represented by SEQ ID NO. 10,
HCDR2 represented by SEQ ID NO. 11,
HCDR3 represented by SEQ ID NO. 12; and
the light chain variable region of the scFv comprises the following three complementarity determining regions (CDRs):
LCDR1 represented by SEQ ID NO. 13,
LCDR2 represented by SEQ ID NO. 14,
LCDR3 represented by SEQ ID NO. 15; or
the heavy chain variable region of the scFv comprises the following three complementarity determining regions (CDRs):
HCDR1 represented by SEQ ID NO. 16,
HCDR2 represented by SEQ ID NO. 17,
HCDR3 represented by SEQ ID NO. 18; and
the light chain variable region of the scFv comprises the following three complementarity determining regions (CDRs):
LCDR1 represented by SEQ ID NO. 19,
LCDR2 represented by SEQ ID NO. 20,
LCDR3 represented by SEQ ID NO. 21.

In the eighth aspect of the present invention, it provides a recombinant immune cell, which expresses an exogenous CAR construct of the seventh aspect of the present invention.

In another preferred embodiment, the immune cell comprises an NK cell, a T cell.

In another preferred embodiment, the immune cell is derived from a human or a non-human mammal (such as a mouse).

In the ninth aspect of the present invention, it provides a pharmaceutical composition, which comprises:
(i) an active ingredient, wherein the active ingredient is selected from the group consisting of: the antibody or the antigen-binding fragment thereof of the first aspect of the present invention, the recombinant protein of the second aspect of the present invention, the antibody conjugate of the sixth aspect of the present invention, the recombinant immune cell of the eighth aspect of the present invention, and a combination thereof; and
(ii) a pharmaceutically acceptable carrier.

In another preferred embodiment, the pharmaceutical composition is a liquid formulation.

In another preferred embodiment, the pharmaceutical composition is an injection.

In another preferred embodiment, the pharmaceutical composition further comprises (iii) other active ingredients, preferably comprising antibodies or antigen-binding fragments thereof that bind to other targets, more preferably comprising an antibody or an antigen-binding fragment thereof that binds to CTLA-4, PD-1, or PD-L1.

In the tenth aspect of the present invention, it provides a method for in vitro detecting CD73 protein in a sample, which comprises the steps of:
(1) contacting the sample in vitro with the antibody of the first aspect of the present invention or the antibody conjugate of the sixth aspect of the present invention;
(2) detecting the formation of an antigen-antibody complex, wherein the formation of a complex indicates the presence of CD73 protein in the sample.

In the eleventh aspect of the present invention, it provides a pharmaceutical combination, which comprises:
(i) a first active ingredient, wherein the first active ingredient comprises the antibody of the first aspect of the present invention, the antibody conjugate of the sixth aspect of the present invention, the recombinant immune cell of the eighth aspect of the present invention, the pharmaceutical composition of the ninth aspect of the present invention, and a combination thereof;
(ii) a second active ingredient, wherein the second active ingredient comprises a secondary antibody or a chemotherapeutic agent.

In another preferred embodiment, the secondary antibody is selected from the group consisting of: a CTLA4 antibody, a PD-1 antibody, a PD-L1 antibody.

In another preferred embodiment, the secondary antibody is a PD-1 antibody.

In another preferred embodiment, the chemotherapeutic agent is selected from the group consisting of: docetaxel, carboplatin, and a combination thereof.

In the twelfth aspect of the present invention, it provides a use of the antibody or the antigen-binding fragment thereof of the first aspect of the present invention, the recombinant protein of the second aspect of the present invention, the antibody conjugate of the sixth aspect of the present invention, the recombinant immune cell of the eighth aspect of the present invention, or the pharmaceutical combination of the twelfth aspect of the present invention, for (a) preparing a reagent or a kit; and/or (b) preparing a medicament for the prevention and/or treatment of CD73-related diseases.

In the thirteenth aspect of the present invention, it provides a method for the preventing and/or treating a CD73-related disease, which comprises the step of: administering the antibody of the first aspect of the present invention, the antibody conjugate of the sixth aspect of the present invention, the recombinant immune cell of the eighth aspect of the present invention, or the pharmaceutical composition of the ninth aspect of the present invention, and a combination thereof, to a subject in need thereof.

In another preferred embodiment, the CD73-related disease is selected from the group consisting of:
hematological cancer, lymphoma, glioblastoma, melanoma, skin cancer, gastric cancer, gastrointestinal stromal tumor, liver cancer, cholangiocarcinoma, gallbladder cancer, peritoneal cancer, colorectal cancer, small intestine cancer, anal cancer, multiple myeloma, pancreatic cancer, breast cancer, ovarian cancer, uterine cancer, cervical cancer, vaginal cancer, bladder cancer, renal cancer, non-small cell lung cancer, small cell lung cancer, prostate cancer, testicular cancer, penile cancer, thyroid cancer, head and neck cancer, esophageal cancer, bone cancer, sarcoma.

It should be understood that within the scope of the present invention, the above-mentioned technical features of the present invention and the technical features specifically described in the following (such as the examples) can be combined with each other to form a new or preferred technical solution, which is not redundantly repeated one by one herein due to space limitation.

### DESCRIPTION OF THE DRAWINGS

FIG. 1 shows the binding ability of murine-derived antibodies to human CD73-His protein.

FIG. 2 shows the inhibitory effect of murine-derived antibodies on CD73 enzyme activity.

FIG. 3 shows the binding activity of chimeric antibodies to human CD73-His protein.

FIG. 4 shows the binding activity of various humanized antibodies derived from 56B10 to human CD73-His protein.

FIG. 5 shows the binding activity of various humanized antibodies derived from 48A11 to human CD73-His protein.

FIG. 6 shows the inhibitory effect of humanized antibodies on human CD73 protein enzyme activity.

FIG. 7 shows the binding activity of humanized antibodies to CD73 protein on the surface of tumor cells (MDA-MB-231 cells).

FIG. 8 shows the binding activity of humanized antibodies to CD73 protein on the surface of tumor cells (H292 cells).

FIG. 9 shows the binding activity of humanized antibodies to CD73 protein on the surface of tumor cells (A375 cells).

FIG. 10 shows the inhibitory effect of humanized antibodies on the enzyme activity of CD73 protein on the surface of tumor cells (MDA-MB-231 cells).

FIG. 11 shows the inhibitory effect of humanized antibodies on the enzyme activity of CD73 protein on the surface of tumor cells (H292 cells).

FIG. 12 shows the inhibitory effect of humanized antibodies on the enzyme activity of CD73 protein on the surface of tumor cells (A375 cells).

FIG. 13 shows that humanized antibodies reversed the inhibition-1 of tumor cell degradation of AMP on CD4⁺ T cell response.

FIG. 14 shows that humanized antibodies reversed the inhibition-2 of tumor cell degradation of AMP on CD8⁺ T cell response.

FIG. 15 shows the in vivo pharmacological activity of humanized antibodies.

FIG. 16 shows the binding epitope determination of humanized antibody 48A11-HuV33 to CD73.

FIG. 17 shows the binding epitope determination of humanized antibody 56B10-HuV31 to CD73.

FIG. 18 shows the affinity-1 of humanized antibody 48A11-HuV33 to various CD73-ND mutant proteins.

FIG. 19 shows the affinity-2 of humanized antibody 48A11-HuV33 to various CD73-ND mutant proteins.

FIG. 20 shows the affinity-3 of humanized antibody 48A11-HuV33 to various CD73-ND mutant proteins.

FIG. 21 shows the affinity-4 of humanized antibody 48A11-HuV33 to various CD73-ND mutant proteins.

FIG. 22 shows the location of key amino acid residues affecting 48A11-HuV33 binding in the 3D structure of CD73 crystallization.

### DETAILED DESCRIPTION

After extensive and intensive research and massive screening, the inventors have obtained an anti-CD73 antibody and a humanized antibody thereof for the first time. The anti-CD73 antibody of the present invention possesses excellent biological activity, which can directly disrupt adenosine-mediated immunosuppression by inhibiting the enzymatic activity of CD73 and blocking the production of adenosine. Specifically, the humanized antibody targeting CD73 of the present invention, when combined with the PD1-targeting antibody, exhibits a synergistic effect that can significantly enhance the therapeutic effect of each drug alone. On this basis, the present invention is completed.

### Terms

In the present invention, the terms “Antibody (abbreviation: Ab)” and “Immunoglobulin G (abbreviation: IgG)” refer to heterotetrasaccharide glycoproteins with identical structural features, which consist of two identical light chains (L) and two identical heavy chains (H). Each light chain is linked to a heavy chain through a covalent disulfide bond, and the number of disulfide bonds differs among different immunoglobulin isotypes. Each heavy chain and light chain also contain regularly spaced intra-chain disulfide bonds. At one end of each heavy chain is a variable region (VH), followed by a constant region. The heavy chain constant region consists of three domains: CH1, CH2, and CH3. Each light chain has a variable region (VL) at one end and a constant region at the other, with the light chain constant region comprising one domain, CL. The constant region of the light chain pairs with the CH1 domain of the heavy chain constant region, and the variable region of the light chain pairs with the variable region of the heavy chain. The constant regions do not directly participate in the binding of the antibody to the antigen, but they exhibit different effector functions, such as participating in antibody-dependent cell-mediated cytotoxicity (ADCC). The heavy chain constant region includes IgG1, IgG2, IgG3, and IgG4 subtypes, while the light chain constant region includes kappa (κ) or lambda (λ). The heavy and light chains of the antibody are covalently linked together by disulfide bonds between the CH1 domain of the heavy chain and the CL domain of the light chain, and the two heavy chains of the antibody are covalently linked together by inter-peptide disulfide bonds formed between hinge regions. The present invention includes not only complete antibodies but also fragments of antibodies with immunological activity or fusion proteins formed by antibodies and other sequences. Therefore, the present invention also includes fragments, derivatives, and analogs of the antibodies. In the present invention, the antibodies can be monospecific, bispecific, trispecific, or more multispecific.

The term “monoclonal antibody” in the present invention refers to an antibody obtained from a substantially homogeneous population, meaning that the individual antibodies contained in this population are identical, except for minor naturally occurring mutations that may be present. Monoclonal antibodies are highly specific to a single antigenic site. Furthermore, unlike conventional polyclonal antibody preparations (which typically contain different antibodies targeting different epitopes), each monoclonal antibody is directed against a single epitope on an antigen. In addition to their specificity, monoclonal antibodies are advantageous because they are synthesized through hybridoma cultures and are not contaminated by other immunoglobulins. The modifier “monoclonal” indicates the nature of the antibody, which is derived from a substantially homogeneous antibody population, and should not be interpreted as requiring the use of any specific method for producing the antibody.

In the present invention, the term “antigen-binding fragment” refers to an active fragment of an antibody that is capable of binding to a specific antigen, preferably a fragment of an antibody that specifically binds to human CD73. Examples of antigen-binding fragments in the present invention include Fab fragments, F(ab′)₂ fragments, Fv fragments, and the like. Fab fragments are produced by digesting antibodies with papain. F(ab′)₂ fragments arc produced by digesting antibodies with pepsin. Fv fragments consist of a tightly non-covalently associated dimer of the heavy chain variable region and light chain variable region of an antibody.

In the present invention, the terms “Fab” and “Fc” refer to the fact that papain can cleave an antibody into two identical Fab fragments and one Fc fragment. The Fab fragment consists of the VH and CH1 domains of the heavy chain of the antibody, as well as the VL and CL domains of the light chain of the antibody. The Fc fragment, also known as the fragment crystallizable (Fc), consists of the CH2 and CH3 domains of the antibody. The Fc fragment has no antigen-binding activity and serves as the site for interaction between the antibody and effector molecules or cells.

In the present invention, the term “scFv” refers to a single chain antibody fragment (scFv), which is typically formed by linking the heavy chain variable region and light chain variable region of the antibody through a linker of 15-25 amino acids.

“Murine-derived antibody” in the present invention refers to an antibody derived from a rat or mouse, preferably a mouse. The murine antibodies of the present invention are obtained by immunizing mice with human CD73 as an antigen and by screening hybridoma cells.

“Chimeric antibody” in the present invention refers to an antibody containing variable region sequences of heavy and light chains from one species and constant region sequences from another species, such as an antibody with mouse heavy and light chain variable regions connected to human constant regions.

In the present invention, the term “variable” refers to the fact that certain portions of the variable region of the antibody differ in sequence, forming the binding and specificity of various specific antibodies to their specific antigens. However, the variability is not uniformly distributed throughout the entire antibody variable region. It is concentrated in three segments called complementarity-determining regions (CDRs) or hypervariable regions within the heavy chain variable region and the light chain variable region. The more conserved portions of the variable region are referred to as framework regions (FRs). The natural variable regions of heavy and light chains each contain four FRs, which generally adopt a β-sheet configuration, connected by three CDRs that form connecting loops, and in some cases, may form partial β-sheet structures. The CDRs in each chain are closely juxtaposed by the FRs and together with the CDRs of the other chain, form the antigen-binding site of the antibody (see Kabat et al., NIH Publ. No. 91-3242, Vol. I, pages 647-669 (1991)).

The term “humanized antibody” in the present invention refers to an antibody whose CDRs are derived from an antibody of a non-human species (preferably mouse), while the remaining portions of the antibody molecule (including the framework regions and the constant regions) are derived from human antibodies. Additionally, framework region residues may be altered to maintain binding affinity.

As used herein, the term “framework region” (FR) refers to the amino acid sequences interposed between CDRs, namely those portions of the variable domains of light and heavy chains of immunoglobulins that are relatively conserved among different immunoglobulins within a single species. Each of the light and heavy chains of an immunoglobulin has four FRs, referred to as FR1-L, FR2-L, FR3-L, FR4-L, and FR1-H, FR2-H, FR3-H, FR4-H, respectively. Accordingly, a light chain variable domain can be denoted as (FR1-L)-(CDR1-L)-(FR2-L)-(CDR2-L)-(FR3-L)-(CDR3-L)-(FR4-L) and a heavy chain variable domain can be denoted as (FR1-H)-(CDR1-H)-(FR2-H)-(CDR2-H)-(FR3-H)-(CDR3-H)-(FR4-H). Preferably, the FRs of the present invention are human antibody FRs or derivatives thereof, which are substantially identical to naturally occurring human antibody FRs, i.e., having a sequence identity of 85%, 90%, 95%, 96%, 97%, 98%, or 99%. Given the amino acid sequences of the CDRs, those skilled in the art can readily determine the framework regions FR1-L, FR2-L, FR3-L, FR4-L, and/or FR1-H, FR2-H, FR3-H, FR4-H.

As used herein, the term “human framework region” refers to a framework region that is substantially identical (about 85% or more, specifically 90%, 95%, 97%, 99%, or 100%) to a naturally occurring human antibody framework region.

In the present invention, the terms “anti”, “bind” and “specifically bind” refer to a non-random binding reaction between two molecules, such as the reaction between an antibody and its target antigen. Typically, the antibody binds to the antigen with an equilibrium dissociation constant (KD) of less than about 10⁻⁷ M, for example, less than about 10⁻⁸ M, 10⁻⁹ M, 10⁻¹⁰ M, 10⁻¹¹ M, or even lower. In the present invention, the term “KD” refers to the equilibrium dissociation constant for a particular antibody-antigen interaction, which describes the binding affinity between the antibody and the antigen. A smaller equilibrium dissociation constant indicates a tighter binding between the antibody and the antigen, and a higher affinity between the antibody and the antigen. For example, the binding affinity of an antibody to an antigen can be determined using Surface Plasmon Resonance (SPR) in a BIACORE instrument or by measuring the relative affinity of antibody-antigen binding using ELISA.

In the present invention, the term “epitope” refers to a polypeptide determinant that specifically binds to an antibody. The epitopes of the present invention are the regions of the antigen that are bound by the antibodies.

In the present invention, the antibodies of the present invention also include conservative variants, which refer to polypeptides formed by replacing up to 10, preferably up to 8, more preferably up to 5, and most preferably up to 3 amino acids with amino acids of similar or close properties compared to the amino acid sequences of the antibodies of the present invention. These conservative variant polypeptides are preferably generated by amino acid substitutions according to Table A.

TABLE A

| Initial residue | Representative substitution | Preferred substitution |
|---|---|---|
| Ala (A) | Val; Leu; Ile | Val |
| Arg (R) | Lys; Gln; Asn | Lys |
| Asn (N) | Gln; His; Lys; Arg | Gln |
| Asp (D) | Glu | Glu |
| Cys (C) | Ser | Ser |
| Gln (Q) | Asn | Asn |
| Glu (E) | Asp | Asp |
| Gly (G) | Pro; Ala | Ala |
| His (H) | Asn; Gln; Lys; Arg | Arg |
| Ile (I) | Leu; Val; Met; Ala; Phe | Leu |
| Leu (L) | Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Leu |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala | Leu |

### Encoding Nucleic Acids and Expression Vectors

The present invention also provides polynucleotide molecules encoding the aforementioned antibodies, or fragments thereof, or fusion proteins. The polynucleotides of the present invention can be in the form of DNA or RNA. DNA forms include cDNA, genomic DNA, or artificially synthesized DNA. The DNA can be single-stranded or double-stranded. The DNA can be the coding strand or the non-coding strand.

The sequences of DNA molecules encoding the antibodies or the fragments thereof of the present invention can be obtained using conventional techniques such as PCR amplification or screening of genomic libraries. In addition, the coding sequences of the light chain and heavy chain can be fused to form a single chain antibody.

Once the relevant sequences are obtained, they can be obtained in large quantities using a recombinant method. This is typically achieved by cloning the sequences into vectors, introducing the vectors into cells, and then isolating the relevant sequences from the proliferated host cells using conventional methods.

Furthermore, relevant sequences can also be synthesized artificially, especially when the fragment length is relatively short. Typically, long fragments can be obtained by first synthesizing multiple small fragments and then linking them together.

At present, the DNA sequences encoding the antibodies (or fragments thereof, or derivatives thereof) of the present invention can be obtained entirely through chemical synthesis. Then, the DNA sequences can be introduced into various existing DNA molecules (or vectors) and cells known in this field. In addition, mutations can be introduced into the protein sequences of the present invention through chemical synthesis.

The present invention also relates to vectors comprising the appropriate DNA sequences as described above, along with appropriate promoters or control sequences. These vectors can be used to transform suitable host cells to enable them to express the proteins.

The vectors mentioned here are conventional expression vectors in the field, referring to expression vectors that comprise appropriate regulatory sequences such as promoter sequences, terminator sequences, polyadenylation sequences, enhancer sequences, marker genes and/or sequences, and other suitable sequences. The expression vectors can be viruses or plasmids, such as suitable phages or phagemids. For more technical details, please refer to, for example, Sambrook et al., Molecular Cloning: A Laboratory Manual, 2nd Edition, Cold Spring Harbor Laboratory Press, 1989. Many known techniques and protocols for nucleic acid manipulation can be found in Current Protocols in Molecular Biology, 2nd Edition, edited by Ausubel et al. The expression vectors of the present invention are preferably pDR1, pcDNA3.1(+), pcDNA3.1/ZEO(+), pDHFR, pcDNA4, pDHFF, pGM-CSF, or pCHO 1.0.

In the present invention, the term “host cell” refers to various conventional host cells in the field, as long as they allow the vector to replicate stably on its own and the polynucleotide molecule carried by it can be effectively expressed. The host cells comprise prokaryotic expression cells and eukaryotic expression cells, and preferably comprise: COS, CHO, NS0, sf9, sf21, DH5α, BL21(DE3), TG1, BL21(DE3), 293F, or 293E cells.

### Preparation of Antibodies

Generally, the transformed host cells obtained are cultured under conditions suitable for the expression of the antibodies of the present invention. Then, the antibodies of the present invention are purified by conventional immunoglobulin purification procedures, such as Protein A-Sepharose, hydroxyapatite chromatography, gel electrophoresis, dialysis, ion exchange chromatography, hydrophobic chromatography, molecular sieve chromatography, affinity chromatography, and other conventional separation and purification methods well known to those skilled in the art.

The obtained monoclonal antibodies can be identified by conventional means. For example, the binding specificity of monoclonal antibodies can be determined by immunoprecipitation or in vitro binding assays such as radioimmunoassay (RIA) or enzyme-linked immunosorbent assay (ELISA). The binding affinity of monoclonal antibodies can be determined by, e.g., Scatchard analysis of Munson et al., Anal. Biochem., 107:220 (1980).

The antibodies of the present invention can be expressed within cells, on cell membranes, or secreted outside the cells. If necessary, the recombinant proteins can be separated and purified by various separation methods utilizing their physical, chemical, and other properties. These methods are well known to those skilled in the art. Examples of these methods include, but are not limited to: conventional renaturation treatment, treatment with protein precipitants (salting-out method), centrifugation, osmotic lysis, ultrasonic treatment, ultracentrifugation, molecular sieve chromatography (gel filtration), adsorption chromatography, ion exchange chromatography, high-performance liquid chromatography (HPLC), and other various liquid chromatography techniques, as well as combinations of these methods.

### Pharmaceutical Compositions and Uses

The present invention also provides a composition. Preferably, the composition is a pharmaceutical composition that comprises the aforementioned antibody, the active fragment thereof, or the fusion protein thereof, along with a pharmaceutically acceptable carrier. Generally, these substances can be formulated in non-toxic, inert, and pharmaceutically acceptable aqueous carrier media, with a pH typically ranging from about 5 to 8, preferably from about 6 to 8, although the pH may vary depending on the nature of the substance being formulated and the disease condition to be treated. Prepared pharmaceutical compositions can be administered through conventional routes, including but not limited to: intravenous injection, intravenous drip, subcutaneous injection, local injection, intramuscular injection, intratumoral injection, intra-abdominal injection (e.g., intraperitoneal), intracranial injection, or intracavitary injection. In the present invention, the term “pharmaceutical composition” refers to a pharmaceutical preparation composition in which the anti-CD73 antibody of the present invention can be combined with a pharmaceutically acceptable carrier to more stably exert therapeutic effects. These formulations can ensure the conformational integrity of the amino acid core sequence of the anti-CD73 antibody disclosed in the present invention, while also protecting the multifunctional groups of the protein from degradation (including but not limited to aggregation, deamination, or oxidation). The pharmaceutical composition of the present invention comprises a safe and effective amount (e.g., 0.001-99 wt %, preferably 0.01-90 wt %, more preferably 0.1-80 wt %) of the aforementioned anti-CD73 antibody (or the conjugate thereof) and pharmaceutically acceptable carriers or excipients. Such carriers include, but are not limited to: saline, buffers, glucose, water, glycerin, ethanol, and combinations thereof. The pharmaceutical formulation should be matched with the administration route. The pharmaceutical composition of the present invention can be made into the form of an injection, for example, prepared by conventional methods using physiological saline or an aqueous solution containing glucose and other adjuvants. Pharmaceutical compositions such as injections and solutions should be manufactured under sterile conditions. The administration amount of the active ingredient is a therapeutically effective amount, such as about 10 micrograms per kilogram of body weight to about 50 milligrams per kilogram of body weight per day. In addition, the anti-CD73 antibody of the present invention can also be used together with other therapeutic agents, such as combined use with other immune molecular modulators (e.g., a CTLA-4 antibody, a PD-1 antibody).

When using the pharmaceutical composition, a safe and effective amount of the anti-CD73 antibody or the immune conjugate thereof is administered to a mammal, wherein the safe and effective amount is generally at least about 10 micrograms per kilogram of body weight, and in most cases does not exceed about 50 milligrams per kilogram of body weight. Preferably, the dosage ranges from about 10 micrograms per kilogram of body weight to about 10 milligrams per kilogram of body weight. Of course, the specific dosage should also consider factors such as the administration route and the patient's health condition, which are within the skill of a skilled physician.

### Antibody-Drug Conjugate (ADC)

The present invention also provides an antibody-drug conjugate (ADC) based on the antibody of the present invention.

Typically, the antibody-drug conjugate comprises the antibody and an effector molecule, wherein the antibody is coupled to the effector molecule, preferably by chemical coupling. Among them, the effector molecule is preferably a drug with therapeutic activity. In addition, the effector molecule can be one or more of a toxin protein, a chemotherapeutic drug, a small molecule drug, or a radionuclide.

The coupling between the antibody of the present invention and the effector molecule can be achieved through a coupling agent. Examples of the coupling agent can be any one or several of non-selective coupling agents, coupling agents utilizing carboxyl groups, peptide chains, and coupling agents utilizing disulfide bonds. The non-selective coupling agent refers to a compound that forms a covalent bond between the effector molecule and the antibody, such as glutaraldehyde. The coupling agent utilizing carboxyl groups can be any one or several of cis-aconitic acid anhydride-type coupling agents (such as cis-aconitic acid anhydride) and acylhydrazone-type coupling agents (with the coupling site being an acylhydrazone).

Certain residues on the antibody (such as Cys or Lys) are used to connect to various functional groups, including imaging agents (such as chromophores and fluorophores), diagnostic agents (such as MRI contrast agents and radioisotopes), stabilizers (such as ethylene glycol polymers), and therapeutic agents. The antibody can be conjugated to a functional agent to form an antibody-functional agent conjugate. The functional agent (such as a drug, a detection reagent, or a stabilizer) is conjugated (covalently linked) to the antibody. The functional agent can be directly connected to the antibody or indirectly connected to the antibody through a linker.

Antibodies can be conjugated to drugs to form antibody-drug conjugates (ADCs). Typically, the ADC contains a linker between the drug and the antibody. The linker can be degradable or non-degradable. Degradable linkers are typically easily degraded in an intracellular environment, such as degradation of the linker at the target site, thereby releasing the drug from the antibody. Suitable degradable linkers include, for example, enzyme-degradable linkers, which include peptide group-containing linkers that can be degraded by intracellular proteases (such as lysosomal proteases or endosomal proteases), or sugar linkers, such as glucuronide-containing linkers that can be degraded by glucuronidases. Peptide group linkers can include, for example, dipeptides such as valine-citrulline, phenylalanine-lysine, or valine-alanine. Other suitable degradable linkers include, for example, pH-sensitive linkers (such as linkers that hydrolyze at a pH less than 5.5, such as hydrazone linkers) and linkers that degrade under reduced conditions (such as disulfide bond linkers). Non-degradable linkers typically release the drug under conditions where the antibody is hydrolyzed by proteases.

Before being connected to the antibody, the linker has reactive functional groups capable of reacting with certain amino acid residues, and the connection is achieved through the reactive functional groups. Sulfhydryl-specific reactive functional groups are preferred and include, for example, maleimide compounds, halogenated amides (such as iodinated, brominated, or chlorinated); halogenated esters (such as iodinated, brominated, or chlorinated); halogenated methyl ketones (such as iodinated, brominated, or chlorinated), benzyl halides (such as iodinated, brominated, or chlorinated); vinyl sulfones, pyridine disulfides; mercury derivatives such as 3,6-di-(mercurymethyl)dioxane, with counterions being acetate, chloride, or nitrate; and polymethylene dimethyl sulfide thiosulfonates. The linker can include, for example, maleimide linked to the antibody via thiosuccinimide.

The drug can be any cytotoxic, cell growth-inhibiting, or immunosuppressive drug. In embodiments, the linker connects the antibody and the drug, and the drug has a functional group capable of bonding with the linker. For example, the drug can have an amino group, a carboxyl group, a sulfhydryl group, a hydroxyl group, or a ketone group that can bond with the linker. In cases where the drug is directly connected to the linker, the drug has a reactive functional group before being connected to the antibody.

Useful drug classes include, for example, anti-tubulin agents, DNA minor groove binders, DNA replication inhibitors, alkylating agents, antibiotics, folate antagonists, antimetabolites, chemosensitizers, topoisomerase inhibitors, vinca alkaloids, and the like. In the present invention, the drug-linker may be used to form an ADC in a single step. In other embodiments, the bifunctional linker compound may be used to form an ADC in a two-step or multi-step process. For example, a cysteine residue reacts with a reactive moiety of the linker in a first step, and in a subsequent step, a functional group on the linker reacts with the drug to form an ADC.

Generally, the functional group on the linker is chosen to facilitate specific reaction with a suitable reactive group on the drug moiety. As non-limiting examples, azide-based moieties can be used to specifically react with reactive alkyne groups on the drug moieties. The drug is covalently attached to the linker through 1,3-dipolar cycloaddition between azide and alkyne. Other useful functional groups include, for example, ketones and aldehydes (suitable for reaction with hydrazides and alkoxyamines), phosphines (suitable for reaction with azides); isocyanates and isothiocyanates (suitable for reaction with amines and alcohols); and activated esters, such as N-hydroxysuccinimide esters (suitable for reaction with amines and alcohols). These and other conjugation strategies, e.g., as described in “Bioconjugate Techniques”, 2nd Edition (Elsevier), are well known to those skilled in the art. It will be understood by those skilled in the art that for selective reaction of the drug moiety and the linker, when a complementary pair of reactive functional groups is chosen, each member of the complementary pair can be used on either the linker or the drug.

The present invention also provides a method of preparing an ADC which further comprises: conjugating the antibody with a drug-linker compound under conditions sufficient to form an antibody-conjugate (ADC).

In some embodiments, the method of the present invention comprises: conjugating an antibody with a bifunctional linker compound under conditions sufficient to form an antibody-linker conjugate. In these embodiments, the method of the present invention further comprise: conjugating the antibody-linker conjugate with a drug moiety under conditions sufficient to covalently attach the drug moiety to the antibody through the linker.

In some embodiments, the antibody-drug conjugate (ADC) is as shown in the following molecular formula:

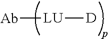

wherein:
Ab represents the antibody,
LU represents the linker,
D represents the drug,
and the subscript p is a value selected from 1 to 8.

### The Main Advantages of the Present Invention Include

(1) The present invention provides a novel CD73 antibody with high affinity for human CD73, which can effectively inhibit CD73 protease activity on the surface of tumor cells.
(2) The CD73 antibody of the present invention can effectively reverse the degradation of AMP by CD73 protein or the inhibition of CD4⁺ and CD8⁺ T cell immune responses mediated by CD73 protein on the surface of tumor cells.
(3) The CD73 antibody of the present invention has good in vivo pharmacological activity, and the combination of CD73 with other immune molecule modulators (such as a CTLA-4 antibody, a PD-1 antibody) can significantly enhance the efficacy of their respective monotherapies, making it a highly promising tumor treatment strategy.

The present invention will be further illustrated below with reference to the specific examples. It should be understood that these examples are only to illustrate the present invention, not to limit the scope of the present invention. The conditions of the experimental methods not specifically indicated in the following examples are usually in accordance with conventional conditions as described in e.g., Sambrook et al., Molecular Cloning: A Laboratory Manual (New York: Cold Spring Harbor Laboratory Press, 1989), or according to the conditions recommended by the manufacturers. Percentages and parts are by weight unless otherwise stated.

### EXPERIMENTAL MATERIALS

Mouse myeloma cell SP2/0: purchased from ATCC, catalog number CRL-1581.
Balb/c mouse: purchased from Shanghai Lingchang Biotechnology Co., Ltd.
H1975 cells: purchased from ATCC, catalog number CRL-1596.
MDA-MB-231 cells: purchased from ATCC, catalog number HTB-26.
H292 cells: purchased from the Cell Bank of the Chinese Academy of Sciences, catalog number SCSP-582.
A375 cells: purchased from the Cell Bank of the Chinese Academy of Sciences, catalog number SCSP-533.
SKOV3 cells: purchased from the Cell Bank of the Chinese Academy of Sciences, catalog number TCHu185.
HRP-goat anti-mouse secondary antibody: purchased from Millipore, catalog number AP181P.
HRP-goat anti-human IgG Fab secondary antibody: purchased from Sigma, catalog number A0293-1ML.
HRP-goat anti-human IgG Fc secondary antibody: purchased from Sigma, catalog number A0170-1ML.
Donkey anti-mouse PE fluorescent secondary antibody: purchased from Jackson, catalog number 715-116-150.
Goat anti-human PE fluorescent secondary antibody: purchased from Jackson, catalog number 109-115-098
FITC-labeled goat anti-human IgG-Fc secondary antibody: purchased from Abcam, catalog number 97224.
TMB: purchased from KPL Company, catalog number 52-00-03.
Bovine serum albumin (BSA): purchased from Sangon Biotech, catalog number A600332-0100.
RPMI 1640 Medium: purchased from Gibco, catalog number 61870127.
Penicillin-streptomycin: purchased from Gibco, catalog number 15140122.
Fetal bovine serum (FBS): purchased from Gibco, catalog number 10091-148.
Polyethylene glycol solution: purchased from Sigma, catalog number P7181.
Hybridoma-SFM: purchased from life technologies, catalog number 12045-076.
HAT: purchased from Gibco, catalog number 21060017.
pcDNA 3.4: purchased from Thermo Fisher, catalog number A14697.
HEK-293F: purchased from Thermo Fisher, catalog number A14527.
Streptavidin (SA): purchased from Sigma, catalog number S0677.
Streptavidin HRP: purchased from BD Pharmingen, catalog number 554066.
EZ-Link NHS-Biotin Reagent: purchased from Thermo Fisher, catalog number 20217.
Cell Titer-Glo: purchased from Promega, catalog number G7570.
AMP: purchased from Sigma, catalog number A1752.
ATP: purchased from Sigma, catalog number A7655.
HBS-EP pH 7.4 buffer: purchased from GE Healthcare, catalog number BR-1006-69.
Protein A/G chip: purchased from GE Healthcare, catalog number BR-1005-30.
CD8 MicroBeads, human: purchased from Miltenyi Biotec, catalog number 130-097-057.
Naive CD4⁺ T Cell Isolation Kit II, human: purchased from Miltenyi Biotec, catalog number 130-094-13.
LS Columns plus tubes: purchased from Miltenyi Biotec, catalog number 130-122-7291.
Ametycin: purchased from Tokyo Chemical Industry, catalog number M2320.
CD3 Monoclonal Antibody (OKT3), Functional Grade: purchased from eBioscience, catalog number 16-0037-85.
CD28 Monoclonal Antibody (CD28.2), Functional Grade: purchased from eBioscience, catalog number 16-0289-85.
Recombinant Human IL-2 Protein: purchased from R&D Systems, catalog number 202-IL-50.
EHNA: purchased from Sigma, catalog number E114.
Purified NA/LE Mouse Anti-Human IFN-γ: purchased from BD Pharmingen, catalog number 554547.
Recombinant Human IFN-γ: purchased from BD Pharmingen, catalog number 554617.
Biotin Mouse Anti-Human IFN-γ: purchased from BD Pharmingen, catalog number 554550.

### Example 1: Antigen Immunization of Animals, Preparation and Screening of Hybridomas

### 1. Antigen Expression

The extracellular domain gene of CD73 (sequence from UniProt, accession number P21589) was constructed into the pcDNA 3.4 expression vector using conventional gene synthesis and molecular cloning methods. In addition, a signal peptide sequence was added to the N-terminus, and a 6×His tag was added to the C-terminus. The vector was then transfected into HEK-293F cells, and after 5 days of expression, the cell culture supernatant was collected and purified to obtain CD73-His protein. Similarly, by replacing the 6×His tag with the Fc sequence of human IgG1, the vector was transfected into HEK-293F cells, and after expression and purification, CD73-Fc protein was obtained.

Amino acid sequence of the extracellular domain of CD73 (SEQ ID NO. 22):

WELTILHTNDVHSRLEQTSEDSSKCVNASRCMGGVARLFTKVQQIRRAEP
NVLLLDAGDQYQGTIWFTVYKGAEVAHFMNALRYDAMALGNHEFDNGVEG
LIEPLLKEAKFPILSANIKAKGPLASQISGLYLPYKVLPVGDEVVGIVGY
TSKETPFLSNPGTNLVFEDEITALQPEVDKLKTLNVNKIIALGHSGFEMD
KLIAQKVRGVDVVVGGHSNTFLYTGNPPSKEVPAGKYPFIVTSDDGRKVP
VVQAYAFGKYLGYLKIEFDERGNVISSHGNPILLNSSIPEDPSIKADINK
WRIKLDNYSTQELGKTIVYLDGSSQSCRFRECNMGNLICDAMINNNLRHT
DEMFWNHVSMCILNGGGIRSPIDERNNGTITWENLAAVLPFGGTFDLVQL
KGSTLKKAFEHSVHRYGQSTGEFLQVGGIHVVYDLSRKPGDRVVKLDVLC
TKCRVPSYDPLKMDEVYKVILPNFLANGGDGFQMIKDELLRHDSGDQDIN
VVSTYISKMKVIYPAVEGRIK

### 2. Antigen Immunization of Mice

Balb/c mice were immunized conventionally with CD73-His protein. On day 1, soluble human CD73-His protein emulsified with Freund's complete adjuvant was injected subcutaneously into Balb/c mice at multiple points (CD73-His protein, 100 μg/mouse/0.5 mL). On day 14, soluble CD73-His protein emulsified with Freund's incomplete adjuvant was injected subcutaneously into Balb/c mice (CD73-His protein, 50 μg/mouse/0.5 mL). On day 28, soluble CD73-His protein emulsified with Freund's incomplete adjuvant was injected subcutaneously into Balb/c mice (CD73-His protein, 50 μg/mouse/0.5 mL). Three weeks later, soluble CD73-His protein (50 μg/mouse/0.2 mL) was injected intraperitoneally to boost the immune response. Three to four days later, the mouse spleens were taken for fusion experiments.

### 3. Preparation and Screening of Hybridomas

Three to four days after the last immunization of mice, a conventional hybridoma technique was employed to fuse mouse spleen cells with mouse myeloma cells SP2/0 using PEG. The fused cells were evenly suspended in a complete culture medium, which was composed of RPMI1640-GLUMAX, 1% Penicillin-streptomycin, 20% FBS, and 1*HAT. The fused cells were plated at 3*10⁴ cells/200 μl/well on 62 96-well cell culture plates and cultured in an incubator. After 7-12 days, the supernatant was collected and screened for hybridoma wells with positive human CD73 binding activity using the ELISA method.

The ELISA method for screening hybridoma wells with positive human CD73 binding activity was as follows: CD73-Fc was diluted to 1 μg/ml with PBS buffer and 100 μl/well was added to the ELISA plates and the plates were incubated overnight at 4° C. The next day, the supernatant was discarded, and the plates were washed once with PBST. Then, 5% skimmed milk powder prepared with PBS was added and the plates were blocked for 2 h at 37° C. The plate were washed three times with PBST and readied for use. The collected hybridoma supernatant was added to the blocked ELISA plates sequentially at 100 μl/well and the plates were incubated for 1 h at 37° C. The plates were washed three times with PBST, then HRP-labeled goat anti-mouse IgG secondary antibody was added and the plates were incubated for 30 min at 37° C. After washing the plates three times with PBST, the residual droplets were patted dry on absorbent paper as much as possible. Then, 100 μl of TMB was added to each well, and the plates were incubated in the dark at room temperature for 5 min for color development. Subsequently, 50 μl of 2M H₂SO₄ stop solution was added to each well to terminate the substrate reaction. The OD values were read at 450 nm using a multimode microplate reader to analyze the binding ability of the tested antibody to the target antigen CD73. Through screening, a total of 30 hybridoma cell lines were obtained. The 30 hybridoma cell lines obtained through amplification and screening in serum-containing complete medium were centrifuged and replaced with serum-free Hybridoma-SFM medium to achieve a cell density of 1˜2×10⁷/ml. They were cultured for 1 week under conditions of 8% CO₂ and 37° C. The culture supernatant was centrifuged and purified using Protein G affinity chromatography to obtain various murine-derived monoclonal antibody proteins against human CD73, which were named respectively.

### Example 3: Binding Ability of Murine-Derived Antibodies to Human CD73-His Protein

Indirect Enzyme-Linked Immunosorbent Assay (ELISA) for Measuring the Binding Ability of Murine-Derived Antibodies to Human CD73-His Protein. The specific method was as follows:

The ELISA plates were coated with SA protein diluted to 1.5 μg/mL in coating buffer (50 mM carbonate coating buffer, pH 9.6) and incubated overnight at 4° C. The supernatant was discarded, and the plates were washed three times with PBST. Then, 5% skimmed milk powder prepared with PBS was added for blocking and the plates were incubated for 2 h at 37° C. After washing the plates once with PBST, CD73-biotin protein (prepared by biotinylating CD73-His protein according to the EZ-Link NHS-Biotin Reagent instructions) was diluted to 0.5 μg/mL and added at 100 μL/well for incubation at room temperature for 1 h. The plates were washed three times with PBST, and the prepared murine-derived monoclonal antibodies against human CD73 were serially diluted with 1% BSA buffer prepared with PBST and added to the ELISA plates at 100 μl/well. The plates were incubated at 37° C. for 1 h. After washing the plates three times with PBST, HRP-labeled goat anti-mouse IgG secondary antibody was added and the plates were incubated for 30 min at 37° C. After washing the plates three times with PBST, the residual droplets were patted dry on absorbent paper as much as possible. Then, 100 μl of TMB color developing solution was added to each well, and the plates were incubated in the dark at room temperature for 5 min for color development. Subsequently, 50 μl of 2M H₂SO₄ stop solution was added to each well to terminate the substrate reaction. The OD values were read at 450 nm using a multimode microplate reader to analyze the binding ability of the tested antibodies to the target antigen human CD73-His.

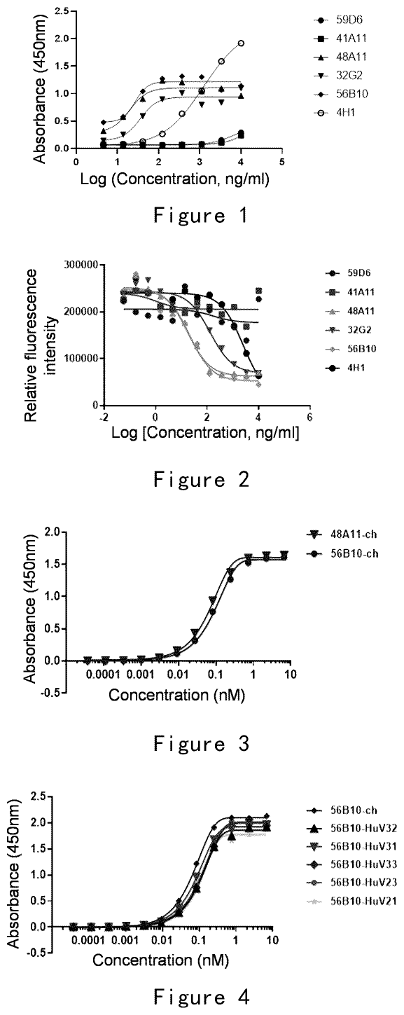

Representative experimental results are shown in FIG. 1 and Table 1, indicating that murine-derived antibodies 48A11 and 56B10 have relatively strong binding activity to CD73 protein.

TABLE 1
EC₅₀ Values of Various Murine-Derived Monoclonal
Antibodies Binding to CD73-Fc

| Sample | 59D6 | 41A11 | 48A11 | 32G2 | 56B10 | 4H1 |
|---|---|---|---|---|---|---|
| EC₅₀ (ng/mL) | 6114 | 148673 | 19.68 | 35.64 | 26.87 | 1235 |

### Example 4: Ability of Murine-Derived Antibodies to Inhibit CD73 Enzyme Activity

CD73 is an enzyme that catalyzes the dephosphorylation of adenosine monophosphate (AMP) to adenosine. Here, the method for detecting ATP was used to measure the inhibitory effect of murine-derived antibodies on CD73 protease activity on the surface of H1975 cells. The specific method was as follows:

H1975 cells in the logarithmic growth phase were collected, and the cell culture medium was removed by centrifugation. The cells were washed once with PBS buffer. The cells were counted and diluted to 3*10⁴/well with RPMI-1640 medium containing 10% FBS, then plated in a 96-well cell culture plate at 100 μL/well and incubated overnight at 37° C. in a cell culture incubator. The next day, the cell culture supernatant was discarded, and the antibodies to be tested were diluted to 10 μg/ml with RPMI-1640 medium, followed by a 5-fold serial dilution. Then, 50 μL/well of the diluted antibodies were added to the cell culture plate and incubated at 37° C. for 30 min. Then, 800 μM of AMP was added at 50 μL/well, and the plate was incubated at 37° C. for 3 h. Next, 25 μL of the culture supernatant was mixed with 25 μL of 80 μM ATP in a 96-well white opaque detection plate, and 50 μL of Cell Titer-Glo detection reagent was added. The mixture was incubated at room temperature for 5 min, and the fluorescence intensity was read and analyzed using a multimode microplate reader.

Representative experimental results are shown in FIG. 2 and Table 2, indicating that compared to other murine-derived monoclonal antibodies, 48A11 and 56B10 exhibit the strongest inhibitory effect on CD73 protease activity on the surface of H1975 cells.

TABLE 2
Inhibitory Effect of Various Murine-Derived
Monoclonal Antibodies on CD73 Protease
Activity on the Surface of H1975 Cells

| Sample | 59D6 | 41A11 | 48A11 | 32G2 | 56B10 | 4H1 |
|---|---|---|---|---|---|---|
| IC₅₀ (ng/mL) | 164.7 | 1.041 | 17.01 | 130.1 | 20.74 | 2507 |

### Example 5: Acquisition of Candidate Antibody Variable Region Genes and Preparation of Chimeric Antibodies

In this example, the heavy chain variable region and light chain variable region of murine-derived antibodies 48A11 and 56B 10 were obtained using molecular biology methods, and chimeric antibodies were further constructed.

RNA was extracted from 48A11 and 56B10 hybridoma cells using Trizol and mRNA reverse transcription was performed to obtain cDNA. Subsequently, using the cDNA as a template, PCR was performed using degenerate primers for murine-derived antibody heavy and light chains (as described in “Antibody Engineering” Volume 1, Edited by Roland Kontermann and Stefan Dübel, with the sequences of the combined primers originating from page 323). The obtained PCR products were sequenced and analyzed using the kabat database to confirm that the obtained sequences were the variable region sequences of the murine-derived antibodies.

The relevant sequence information is as follows:
48A11 heavy chain variable region gene sequence, full-length of 351 bp, encoding 117 amino acid residues, and the nucleotide sequence thereof is (SEQ ID NO. 23):

CAGGTCCAACTGCAGCAGCCTGGGGCTGAACTGGTGAAGCCTGGGGCTTC
AGTGAAGCTGTCCTGCAAGGCTTCTGGCTACACCTTCACCAGCTACTGGA
TGCACTGGGTGAAGCAGAGGCCTGGACAAGGCCTTGAGTGGATTGGAGAG
ATTAATCCTAGCATCGGTCGTACTAACTACAATGAGAAGTTCAAGAGCAA
GGCCACACTGACTGTAGACAAATCCTCCAGCACAGCCTTCATGCAACTCA
GCAGTCTGACATCTGAGGACTCTGCGGTCTATTACTGTGCAAGAAGGGTC
TATGGTACTATGGACTACTGGGGTCAAGGAACCTCAGTCACCGTCTCCTC
A

48A11 heavy chain variable region amino acid sequence (SEQ ID NO. 24):

QVQLQQPGAELVKPGASVKLSCKASGYTFTSYWMHWVKQRPGQGLEWIG
EINPSIGRTNYNEKFKSKATLTVDKSSSTAFMQLSSLTSEDSAVYYCAR
RVYGTMDYWGQGTSVTVSS

48A11 light chain variable region gene sequence, full-length of 318 bp, encoding 106 amino acid residues, and the nucleotide sequence thereof is (SEQ ID NO. 25):

GACATCAAGATGACCCAGTCTCCATCTTCCATGTATGCATCTCTAGGAGA
GAGAGTCACTATCACTTGCAAGGCGAGTCAGGACATTAATAGCTATTTAA
GCTGGTTCCAGCAGAAACCAGGGAAATCTCCTAAGACCCTGATCTATCGT
GCAAACATATGGGTAGATGGGGTCCCATCAAGGTTCAGTGGCAGTGGATC
TGGGCAAGATTATTCTCTCACCATCAGCAGCCTGGAGTATGAAGATATGG
GAATTTATTATTGTCTACAGTATGATGAGTTATACACGTTCGGAGGGGGG
ACCAAGCTGGAAATAAAA

48A11 light chain variable region amino acid sequence (SEQ ID NO. 26):

DIKMTQSPSSMYASLGERVTITCKASQDINSYLSWFQQKPGKSPKTLIY
RANIWVDGVPSRFSGSGSGQDYSLTISSLEYEDMGIYYC
LQYDELYTFGGGTKLEIK

56B10 heavy chain variable region gene sequence, full-length of 357 bp, encoding 119 amino acid residues, and the nucleotide sequence thereof is (SEQ ID NO. 27):

CAGGTCCAGCTGCAGCAGTCTGGACCTGAGCTGGTGAAGCCTGGGGCTTC
AGTGAGGATATCCTGCAAGACTTCTGGCTACACCTTCACAAGATACTATA
TATATTGGGTGAAGCAGAGGCCTGGACAGGGACTTGAGTGGATTGGATGG
ATTTATCCTGGAAATTTTAATACTAAGTACAATGAGAAGTTCAAGGGCAA
GGCCACACTGACTGCAGACACATCCTCCAGCACAGCCTACATGCAGCTCA
GCAGCCTGACCTCTGAGGACTCTGCGGTCTATTTCTGTGCAAGAGATGTA
TATGATTACGCGGGATTTGCTTACTGGGGCCAGGGGACTCTGGTCACTGT
CTCTGCA

56B10 heavy chain variable region amino acid sequence (SEQ ID NO. 28):

QVQLQQSGPELVKPGASVRISCKTSGYTFTRYYIYWVKQRPGQGLEWIG
WIYPGNFNTKYNEKFKGKATLTADTSSSTAYMQLSSLTSEDSAVYFCAR
DVYDYAGFAYWGQGTLVTVSA

56B10 light chain variable region gene sequence, full-length of 321 bp, encoding 107 amino acid residues, and the nucleotide sequence thereof is (SEQ ID NO. 29):

GACATTGTGATGACCCAGTCTCACAGATTCTTGTCCACATCAGTAGGAGA
CAGGGTCAGCATCACCTGCAAGGCCAGTCAGGGTGTGGCTACTGCTGTTG
CCTGGTATCAACAGAAACCAGGACAATCTCCTAAACTCCTGATTTACTGG
GCATCCACCCGGCACACTGGAGTCCCTGATCGCTTCACAGGCAGTGGATC
TGGGACAGATTTCACTCTCACCATTAGCAATGTGCAGTCTGAAGACTTGG
CAGATTATTTCTGTCAGCAATATAGCAGCTATCCGTGGACGTTCGGTGGA
GGCACCAAGCTGGAAATCAAA

56B10 light chain variable region amino acid sequence (SEQ ID NO. 30):

DIVMTQSHRFLSTSVGDRVSITCKASQGVATAVAWYQQKPGQSPKLLIY
WASTRHTGVPDRFTGSGSGTDFTLTISNVQSEDLADYFC
QQYSSYPWTFGGGTKLEIK

The obtained heavy chain variable region sequences of the hybridomas were spliced with the human IgG4 (S228P) constant region (amino acid sequence SEQ ID NO. 31), and the light chain variable region sequences were spliced with the human kappa chain constant region (amino acid sequence SEQ ID NO. 32). The heavy and light chains of each chimeric antibody were constructed into the pcDNA3.4 expression vector, and then transfected into HEK-293F cells for expression and purification. The obtained chimeric antibodies were named 48A11-ch and 56B 10-ch, respectively.

### Example 6: Binding Activity of Chimeric Antibodies to Human CD73-His Protein

Refer to Example 3 to determine the binding affinity of chimeric antibodies 48A11-ch and 56B 10-ch to human CD73-his protein. The experimental results, as shown in FIG. 3, showed that 48A11-ch and 56B10-ch had an EC50 of 0.067 nM and 0.09 nM for binding to human CD73-his protein, respectively.

Refer to Example 3 to determine the binding affinity of chimeric antibodies 48A11-ch and 56B 10-ch to human CD73-his protein. The experimental results, as shown in FIG. 3, showed that 48A11-ch and 56B10-ch had an EC50 of 0.067 nM and 0.09 nM for binding to human CD73-his protein, respectively.

### Example 7: Construction and Preparation of Humanized Antibodies

The amino acid sequences of the light chain variable region and heavy chain variable region of each candidate murine-derived antibody were analyzed. Based on the Kabat rules, the antigen complementarity-determining regions (CDRs) and four framework regions (FRs) of the murine-derived antibodies 48A11 and 56B10 were identified. Among them, the amino acid sequences of the 48A11 heavy chain complementarity-determining regions are:
HCDR1: SYWMH SEQ ID NO.10
HCDR2: EINPSIGRTNYNEKFKS SEQ ID NO.11
HCDR3: RVYGTMDY SEQ ID NO.12
the amino acid sequences of the light chain complementarity-determining regions are:
LCDR1: KASQDINSYLS SEQ ID NO.13
LCDR2: RANIWVD SEQ ID NO.14
LCDR3: LQYDELYT SEQ ID NO.15
the amino acid sequences of the 56B10 heavy chain complementarity-determining regions are:
HCDR1: RYYIY SEQ ID NO.16
HCDR2: WIYPGNFNTKYNEKFKG SEQ ID NO.17
HCDR3: DVYDYAGFAY SEQ ID NO.18
the amino acid sequences of the light chain complementarity-determining regions are:
LCDR1: KASQGVATAVA SEQ ID NO.19
LCDR2: WASTRHT SEQ ID NO.20
LCDR3: QQYSSYPWT SEQ ID NO.21

From the Germline database, the best-matched humanization templates were selected for the non-FR regions of each murine-derived antibody mentioned above. Then, the CDR regions of the murine-derived antibodies were grafted onto the selected humanization templates, replacing the CDR regions of the human templates. The heavy chain variable regions were recombined with the human IgG4 constant region, and the light chain variable regions were recombined with the human kappa chain constant region. Additionally, based on the three-dimensional structure of the antibodies, the buried residues, residues with direct interactions with the CDRs, and residues critical for maintaining the conformation of the VL and VH of each antibody were subjected to back mutations. This process ultimately yielded multiple humanized antibodies, with their corresponding heavy chain, light chain variable regions, and sequences shown in Table 3. The heavy and light chains of each humanized antibody were constructed into the pcDNA3.4 expression vector, transfected into HEK-293F cells for expression and purification to obtain the humanized antibodies.

The relevant sequence information obtained is as follows:

TABLE 3
Sequence Listing of Variable Region
Sequences for Each Humanized Antibody

| | | Amino Acid | Nucleotide |
|---|---|---|---|
| Antibody | | Sequence | Sequence |
| 56B10-HuV21 | VH | SEQ ID NO: 1 | SEQ ID NO: 33 |
| | VL | SEQ ID NO: 2 | SEQ ID NO: 34 |
| 56B10-HuV23 | VH | SEQ ID NO: 1 | SEQ ID NO: 33 |
| | VL | SEQ ID NO: 3 | SEQ ID NO: 35 |
| 56B10-HuV31 | VH | SEQ ID NO: 4 | SEQ ID NO: 36 |
| | VL | SEQ ID NO: 2 | SEQ ID NO: 34 |
| 56B10-HuV32 | VH | SEQ ID NO: 4 | SEQ ID NO: 36 |
| | VL | SEQ ID NO: 5 | SEQ ID NO: 37 |
| 56B10-HuV33 | VH | SEQ ID NO: 4 | SEQ ID NO: 36 |
| | VL | SEQ ID NO: 3 | SEQ ID NO: 35 |
| 48A11-HuV22 | VH | SEQ ID NO: 6 | SEQ ID NO: 38 |
| | VL | SEQ ID NO: 7 | SEQ ID NO: 39 |
| 48A11-HuV23 | VH | SEQ ID NO: 6 | SEQ ID NO: 38 |
| | VL | SEQ ID NO: 8 | SEQ ID NO: 40 |
| 48A11-HuV33 | VH | SEQ ID NO: 9 | SEQ ID NO: 41 |
| | VL | SEQ ID NO: 8 | SEQ ID NO: 40 |

The relevant amino acid sequences are as follows:

SEQ ID NO: 1
QVQLVQSGAEVKKPGASVKVSCKTSGYTFTRYYIYWVRQAPGQGLEWIG
WIYPGNFNTKYNEKFKGRATLTADTSASTAYMELSSLRSEDTAVYYCAR
DVYDYAGFAYWGQGTLVTVSS
SEQ ID NO: 2
DIQLTQSPSFLSASVGDRVTITCKASQGVATAVAWYQQKPGQSPKLLIY
WASTRHTGVPDRFSGSGSGTEFTLTISSLQPEDFATYYC
QQYSSYPWTFGQGTKVEIK
SEQ ID NO: 3
DIQMTQSPSFLSASVGDRVTITCKASQGVATAVAWYQQKPGKSPKLLIY
WASTRHTGVPSRFSGSGSGTEFTLTISSLQPEDFATYFC
QQYSSYPWTFGQGTKVEIK
SEQ ID NO: 4
QVQLQQSGAEVKKPGASVKVSCKASGYTFTRYYIYWVRQRPGQGLEWIG
WIYPGNFNTKYNEKFKGRATLTADTSASTAYMELSSLTSEDTAVYYCAR
DVYDYAGFAYWGQGTLVTVSS
SEQ ID NO: 5
DIQLTQSPSFLSASVGDRVTITCKASQGVATAVAWYQQKPGKAPKLLIY
WASTRHTGVPSRFSGSGSGTEFTLTISSLQPEDFATYYC
QQYSSYPWTFGQGTKVEIK
SEQ ID NO: 6
QVQLVQSGAEVKKPGASVKVSCKASGYTFTSYWMHWVRQAPGQGLEWIG
EINPSIGRTNYNEKFKSRATLTVDKSTSTAYMELSSLRSEDTAVYYCAR
RVYGTMDYWGQGTLVTVSS
SEQ ID NO: 7
DIQMTQSPSSLSASVGDRVTITCKASQDINSYLSWFQQKPGKSPKTLIY
RANIWVDGVPSRFSGSGSGQDYTFTISSLQPEDIATYYC
LQYDELYTFGQGTKVEIK
SEQ ID NO: 8
DIQMTQSPSSLSASVGDRVTITCKASQDINSYLSWFQQKPGKSPKLLIY
RANIWVDGVPSRFSGSGSGQDYTFTISSLQPEDIATYYC
LQYDELYTFGQGTKVEIK
SEQ ID NO: 9
QVQLVQSGAEVKKPGASVKVSCKASGYTFTSYWMHWVRQAPGQGLEWIG
EINPSIGRTNYNEKFKSRVTLTRDTSTSTAYMELSSLRSEDTAVYYCAR
RVYGTMDYWGQGTLVTVSS

### Example 8: Binding Activity of Humanized Antibodies to Human CD73-His Protein

The binding affinity of various humanized antibodies to human CD73-his protein was measured with reference to Example 3.

The experimental results are shown in FIG. 4 and Table 4, indicating that the binding affinity of each humanized antibody of 56B10 to CD73 protein is comparable to that of chimeric antibody 56B10-ch.

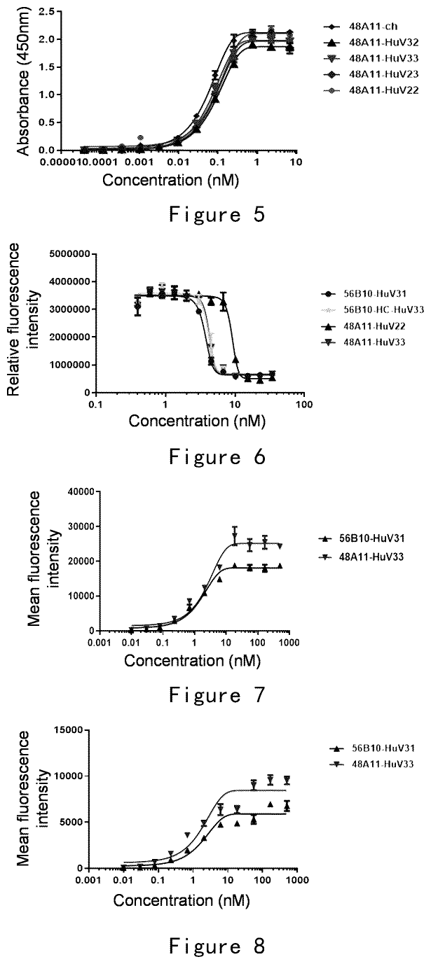

As shown in FIG. 5 and Table 5, the binding affinity of each humanized antibody of 48A11 to CD73 protein is comparable to that of chimeric antibody 48A11-ch.

TABLE 4
EC₅₀ Values of Binding of 56B10 Humanized
Antibodies to CD73 Protein

| Sample | EC₅₀ (nM) |
|---|---|
| 56B10-ch | 0.070 |
| 56B10-HuV32 | 0.105 |
| 56B10-HuV31 | 0.079 |
| 56B10-HuV33 | 0.112 |
| 56B10-HuV23 | 0.107 |
| 56B10-HuV21 | 0.110 |

TABLE 5
EC₅₀ Values of Binding of 48A11 Humanized
Antibodies to CD73 Protein

| Sample | EC₅₀ (nM) |
|---|---|
| 48A11-ch | 0.058 |
| 48A11-HuV32 | 0.087 |
| 48A11-HuV33 | 0.069 |
| 48A11-HuV23 | 0.081 |
| 48A11-HuV22 | 0.093 |

### Example 9: Inhibition of Humanized Antibodies on Human CD73 Protease Activity

CD73-His protein was diluted to 0.5 μg/mL in Tris-MgCl₂ solution (25 mM Tris, 5 mM MgCl₂, pH=7.5) and added to a white 96-well cell culture plate at 25 μL per well. The antibodies were serially diluted in Tris-MgCl₂ solution and added to the 96-well plate at 25 μL per well, followed by incubation at 37° C. for 1 hour.

25 μL of a pre-mixed AMP/ATP solution (with final concentrations of 300 μM AMP and 100 μM ATP) was added to each well and the plate was incubated at 37° C. for 1 hour. Then, 75 μL of Cell Titer-Glo detection reagent was added to each well, and the mixture was shaken for 2 minutes before reacting at room temperature for 5-8 minutes. Subsequently, the 96-well plate was placed in a multimode microplate reader to measure the fluorescence intensity, and the inhibitory effect of each antibody on CD73 protease activity was calculated.

The experimental results, as shown in FIG. 6 and Table 6, indicate that 48A11-HuV22 exhibits the weakest inhibitory effect on CD73 protease's ability to degrade AMP, while 56B10-HuV33 and 48A11-HuV33 have comparable inhibitory effects. Among them, 56B10-HuV31 demonstrates the strongest inhibitory effect.

TABLE 6
Inhibitory Effect of Various Humanized
Antibodies on CD73 Protease Activity

| Sample | IC₅₀ (nM) |
|---|---|
| 56B10-HuV31 | 3.660 |
| 56B10-HuV33 | 4.129 |
| 48A11-HuV22 | 9.023 |
| 48A11-HuV33 | 4.274 |

### Example 10: Determination of Binding Kinetics of Humanized Monoclonal Antibodies to CD73

Antibodies to be tested were captured using a chip covalently coupled with Protein A/G, with the following operational parameters: antibody concentration of 2 μg/mL, contact time of 75 seconds, flow rate of 10 μL/min, and regeneration contact time of 30 seconds. The CD73-his antigen was diluted in HBS-EP pH 7.4 buffer, with the highest concentration being 100 nM, and diluted in a 2-fold gradient down to 0.78 nM. Duplicate wells and a 0 concentration point were set up. 6M guanidine hydrochloride solution was used as the regeneration buffer. Using the Biacore 8K, the samples were injected with the following parameters: binding time of 180 seconds, dissociation time of 900 seconds, flow rate of 30 μL/min, and regeneration contact time of 30 seconds. After the procedure was completed, the Biacore 8K Evaluation Software was used to analyze the data according to the “1:1 binding kinetics model,” yielding the binding kinetics parameters of each antibody to CD73.

The results, as shown in Table 7, indicate that the humanized antibodies 56B10-HuV31 and 48A11-HuV33 have similar binding constants (ka), dissociation constants (kd), and equilibrium dissociation constants (KD) for CD73.

TABLE 7
Binding Kinetics Parameters of Various
Humanized Antibodies to CD73

| Sample | ka (1/Ms) | kd (1/s) | KD (M) |
|---|---|---|---|
| 48A11-HuV33 | 1.68E+05 | 3.81E−04 | 2.26E−09 |
| 56B10-HuV31 | 1.99E+05 | 3.76E−04 | 1.89E−09 |

### Example 11: Binding Activity of Humanized Antibodies to CD73 Protein on the Surface of Tumor Cells

MDA-MB-231 cells (triple-negative breast cancer), H292 cells (human lung cancer cells), and A375 cells (human malignant melanoma cells), all in their logarithmic growth phase, were collected respectively. The cells were washed once with RPMI-1640 basic medium and then adjusted to a cell concentration of 2.0×10⁶ cells/mL in RPMI-1640 basic medium containing 1% BSA (antibody diluent). The antibodies to be tested were diluted in a 3-fold gradient using the antibody diluent, and 100 μL of each concentration of the test antibodies was mixed with 100 μL of cells in a 96-well cell culture plate and incubated at 4° C. for 1 hour (the maximum working concentration of the test antibodies was 500 nM, with 1×10⁵ target cells per well). The cells were washed twice with PBS to remove unbound test antibodies. Then, the cells were mixed with 200 μL of 5 μg/mL FITC-labeled goat anti-human IgG-Fc secondary antibody and incubated at 4° C. for 30 minutes. The cells were washed twice again with PBS to remove unbound secondary antibodies, and finally, the cells were resuspended in 200 μL of PBS. The binding affinity of the test antibodies to the cells was determined using a flow cytometer.

As shown in FIG. 7, both 56B10-HuV31 and 48A11-HuV33 can bind to MDA-MB-231 cells, and 48A11-HuV33 exhibits a stronger affinity, with EC₅₀ values of 1.387 nM and 1.927 nM, respectively.

As shown in FIG. 8, both 56B10-HuV31 and 48A11-HuV33 can bind to H292 cells, and 48A11-HuV33 exhibits a stronger affinity, with EC₅₀ values of 2.549 nM and 2.305 nM, respectively.

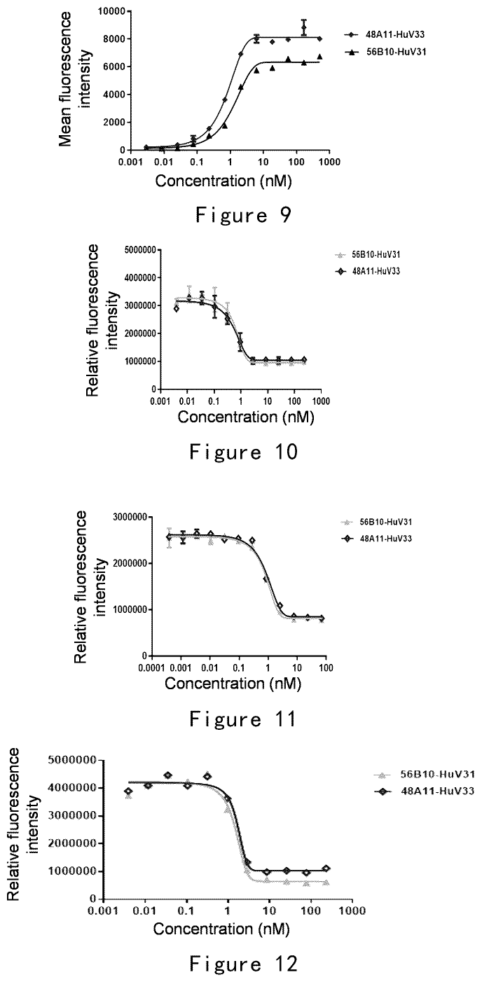

As shown in FIG. 9, both 56B10-HuV31 and 48A11-HuV33 can bind to A375 cells, and 48A11-HuV33 exhibits a stronger affinity, with EC₅₀ values of 1.269 nM and 0.789 nM, respectively.

### Example 12: Inhibitory Effect of Humanized Antibodies on CD73 Protease Activity on the Surface of Tumor Cells

MDA-MB-231, H292, and A375 cells, all in their logarithmic growth phase, were collected respectively. The cells were washed once with RPMI-1640 basic medium and adjusted to cell concentrations of 1×10⁵ cells/mL, 1×10⁵ cells/mL, and 1×10⁶ cells/mL, respectively, in RPMI-1640 basic medium containing 10% FBS. A 96-well cell culture plate was prepared with 100 μL of cell suspension per well and cultured overnight at 37° C. in a cell incubator. The next day, the cell supernatant was discarded, and the cells were washed once with Tris-MgCl₂ solution. The test antibodies were serially diluted in Tris-MgCl₂ solution in a 3-fold gradient, and 50 μL of each concentration of the test antibodies was added to each cell culture well. The cells were incubated at 37° C. for 0.5 hours (the maximum working concentration of the test antibodies was 100 μg/mL). AMP was added to the cell culture plate to a final concentration of 300 μM, with 50 μL added to each well, and the cells were further incubated at 37° C. for 3 hours. Then, 50 μL of the cell incubation supernatant was transferred to a 96-well white cell culture plate, and an equal volume of ATP was added. For MDA-MB-231 cells, the final concentration of ATP was 100 μM (ATP: AMP=1:3), while for H292 and A375 cells, the final concentration of ATP was 60 μM (ATP: AMP=1:5). The white cell culture plate was then incubated at 37° C. for 15 minutes. An equal volume of Cell Titer-Glo reagent was added and allowed to react for 10 minutes. The fluorescence values were read on a multimode microplate reader to analyze the inhibitory effect of each sample on CD73 protease activity on the surface of tumor cells.

As shown in FIG. 10, both 56B10-HuV31 and 48A11-HuV33 can effectively inhibit the activity of CD73 protease on the surface of MDA-MB-231 cells in degrading AMP, with IC₅₀ values of 0.610 nM and 0.566 nM, respectively.

As shown in FIG. 11, both 56B10-HuV31 and 48A11-HuV33 can effectively inhibit the activity of CD73 protease on the surface of H292 cells in degrading AMP, with IC₅₀ values of 0.818 nM and 0.859 nM, respectively.

As shown in FIG. 12, both 56B10-HuV31 and 48A11-HuV33 can effectively inhibit the activity of CD73 protease on the surface of A375 cells in degrading AMP, with IC₅₀ values of 1.339 nM and 1.482 nM, respectively.

### Example 13: Humanized Antibody Reverses the Inhibition of CD4⁺ and CD8⁺ T Cell Responses by Tumor Cell-mediated AMP Degradation

### 1. T Cell Sorting

Freshly purchased PBMC cells (purchased from Shanghai Saili Biotechnology Co., Ltd.) were placed on ice, counted, and sorted into CD8⁺ T and CD4⁺ T cells respectively, according to the instructions for magnetic bead sorting of CD8⁺ T and CD4⁺ T cells, for later use.

### 2. Tumor Cell Treatment

H292 cells in the logarithmic growth phase were taken, and the cell culture supernatant was discarded. Freshly prepared RPMI-1640 complete medium containing 50 μg/mL Ametycin was added, and the cells were continuously cultured at 37° C. in a cell incubator for 3 hours.

### 3. Co-culture of Tumor Cells and T Cells

The sorted CD8⁺ T and CD4⁺ T cells were seeded into 96-well cell culture plates pre-coated with 1 μg/mL and 3 μg/mL CD3 antibodies, respectively, at a density of 5*10⁴/100 μL/well. Then, 3 μg/mL of CD28 antibody and IL-2 (working concentration of 100 IU/mL) prepared in RPMI-1640 complete medium were added to activate the T cells. EHNA with a final concentration of 0.5 μM and AMP with a final concentration of 300 μM were added to each experimental well. Further, varying concentrations of the test antibodies and the Ametycin-treated H292 cells were added to each cell culture well at a cell density of 2.5*10⁴/50 μL/well. The 96-well cell culture plates were placed in a cell incubator for 4 days, and the cell culture supernatant was collected for IFN-γ detection.

### 4. IFN-γ Detection

The mouse anti-human IFN-γ antibody was diluted to 1 μg/mL with ELISA coating buffer and coated on the ELISA plate at 100 μL/well. The plate was placed in a wet box at 4° C. for 16 hours. The ELISA plate was washed three times with PBST to remove unbound antigens and patted dry on absorbent paper to remove excess liquid. Then, 2% BSA prepared in PBS was added to the plate at 200 μL/well and incubated at room temperature for 2 hours for blocking. After washing once with PBST to remove excess blocking solution and patting dry, the cell supernatants treated with varying concentrations of antibodies were added at 100 μL/well and incubated at room temperature for 1 hour. The ELISA plate was washed three times with PBST, and biotin-mouse anti-human IFN-γ antibody diluted to 1 μg/mL was added to the plate at 100 μL/well and incubated at room temperature for 1 hour. After washing the ELISA plate three times with PBST, HRP-SA diluted 1:5000 was added to the plate at 100 μL/well and incubated at room temperature for 30 minutes. The ELISA plate was washed five times with PBST, patted dry on absorbent paper to remove excess liquid, and TMB color developing solution was added at 100 μL/well. The plate was allowed to develop color to an appropriate degree, and 2M H₂SO₄ was added at 50 μL/well to terminate the color development. The absorbance was measured at a wavelength of 450 nm in a multimode microplate reader, and the data were analyzed.

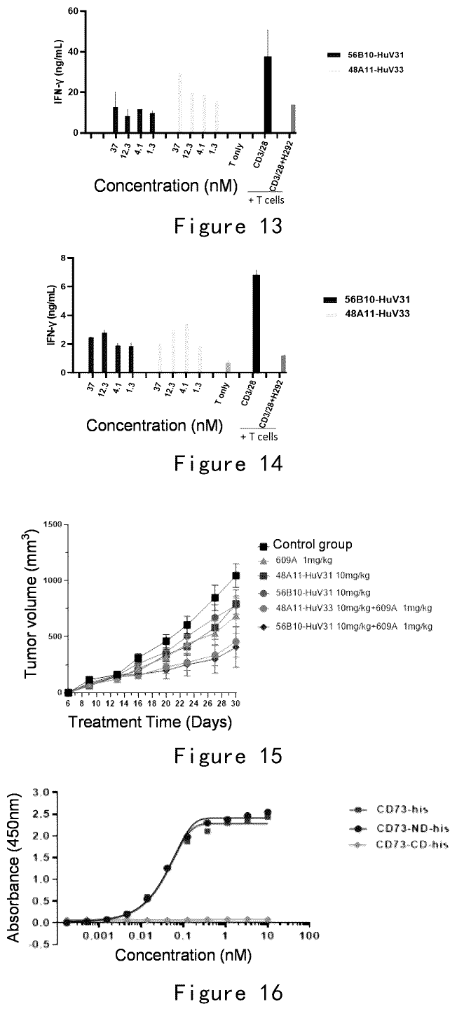

The results, as shown in FIGS. 13 and 14, respectively, indicate that the humanized antibody 48A11-HuV33 can significantly reverse the inhibition of CD4⁺ and CD8⁺ T cell responses by adenosine produced from AMP degradation by tumor cells H292, showing significantly better activity than 56B10-HuV31.

### Example 14: Cross-Reactivity of Humanized Antibodies against CD73 Proteins From Different Species

The experimental method refers to Example 10, with the antigens replaced by cynomolgus monkey CD73 protein (purchased from Sino biological, catalog number 90192-C08H) and mouse CD73 protein (purchased from Sino biological, catalog number 50231-M08H).

The experimental results are shown in Table 8. Both 48A11-HuV33 and 56B10-HuV31 can effectively bind to cynomolgus monkey CD73 protein but not to mouse CD73 protein, indicating that cynomolgus monkeys can be used as a relevant species for subsequent pharmacokinetic and toxicological studies.

TABLE 8
Cross-reactivity of Humanized Antibodies against
CD73 Proteins from Cynomolgus Monkey and Mouse

| Sample | Antigen | ka (1/Ms) | kd (1/s) | KD (M) |
|---|---|---|---|---|
| 48A11-HuV33 | CD73-mouse | \ | \ | \ |
| 56B10-HuV31 | CD73-mouse | \ | \ | \ |
| 48A11-HuV33 | CD73-cynomolgus | 7.28E+04 | 8.83E−04 | 1.21E−08 |
| | monkey | | | |
| 56B10-HuV31 | CD73-cynomolgus | 9.99E+04 | 7.12E−04 | 7.13E−09 |
| | monkey | | | |

\: No binding, no binding signal detected.

### Example 15: Pharmacological Activity of Humanized Antibodies In Vivo

In this example, human peripheral blood mononuclear cells (hPBMC) (purchased from Shanghai Saili Biotechnology Co., Ltd.) were used to reconstruct a human-derived immune system in NSG mice. A human lung cancer NCI-H292 subcutaneous xenograft model was established on these mice to evaluate the in vivo pharmacological activity of candidate antibodies.

The specific implementation steps were as follows: NCI-H292 lung cancer cells cultured in vitro were collected, and the cell suspension concentration was adjusted to 5×10⁷/ml, which was then mixed with Matrigel in a 1:1 ratio. Freshly resuscitated PBMC cells were resuspended in PBS, and the PBMC suspension concentration was adjusted to 1×10⁷/ml. The mixed tumor cell suspension and PBMC suspension were mixed in a 1:1 ratio. Under aseptic conditions, 200 μl of the cell mixture suspension was inoculated subcutaneously into the upper right back of NSG mice. On the same day, mice inoculated with mixed cells were randomly grouped based on their body weight, with 8 mice in each group. The groups included a blank control group; an anti-PD1 monoclonal antibody 609A group (refer to patent application PCT/CN2018/073575), with a dose of 1 mg/kg; anti-CD73 monoclonal antibody groups, including 56B10-HuV31 and 48A11-HuV33, both with a dose of 10 mg/kg; and combination groups of anti-CD73 monoclonal antibodies with 609A, respectively. Treatments were administered intraperitoneally twice a week for a total of 8 doses. Tumor volume was measured twice a week.

The tumor growth curves over time for each group are shown in FIG. 15. At the end of the experiment, compared to the control group, the inhibition rate of 609A monotherapy on H292 subcutaneous xenografts in mice was 34.4%, the inhibition rate of 56B 10-HuV31 was 25.3%, and the inhibition rate of 48A11-HuV33 was 24.4%. When 56B10-HuV31 was combined with 609A, the inhibition rate was 61.2%, and when 48A11-HuV33 was combined with 609A, the inhibition rate was 52.9%. These results indicate that the combination of humanized antibodies targeting CD73, 56B10-HuV31 and 48A11-HuV33, with the PD1-targeting antibody 609A can significantly enhance the efficacy of each monotherapy.

### Example 16: Determination of Binding Epitopes of Humanized Antibodies to CD73

Based on the domain characteristics of the CD73 sequence and the research results of literature (DOI: 10.1016/j.str.2012.10.001), CD73 extracellular domain was expressed in two parts: the N-terminal region from amino acid W at position I to amino acid D at position 291, and the C-terminal region from amino acid Q at position 311 to amino acid S at position 523. The expression and purification were performed according to the method in Example 1, and the above two parts were named CD73-ND-his and CD73-CD-his, respectively.

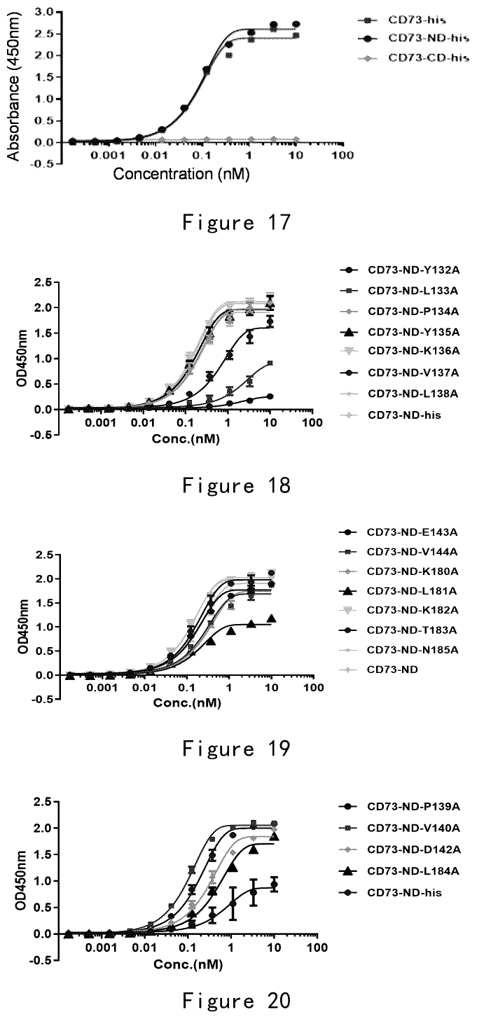

Referring to the experimental method in Example 3, the binding regions of 48A11-HuV33 and 56B10-HuV31 to CD73 protein were determined. The results are shown in FIGS. 16 and 17, respectively. Both 48A11-HuV33 and 56B10-HuV31 only bound to CD73-ND-his and not to CD73-CD-his, indicating that humanized antibodies 48A11-HuV33 and 56B10-HuV31 bind to the N-terminal domain of CD73 protein.

Further, based on the binding characteristics of 48A11-HuV33 and 56B10-HuV31 to CD73 protein, two sequence fragments of the 133rd amino acid Y to 144th amino acid V and the 180th amino acid K to 187th amino acid N in the N-terminal domain of CD73 protein, were selected for alanine scanning site-specific mutagenesis through PCR (polymerase chain reaction). Subsequently, expression and purification were performed according to Example 1 to obtain each CD73-ND mutant.

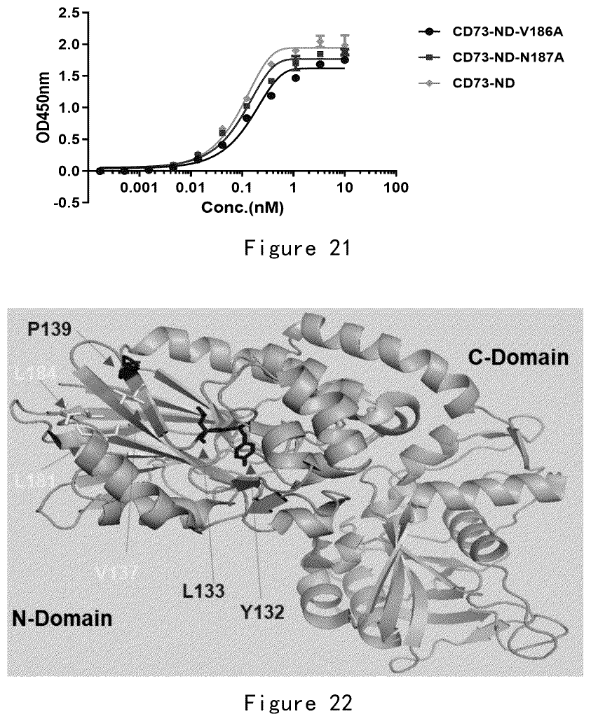

Referring to the experimental method in Example 3, the affinity of 48A11-HuV33 for various mutant proteins of CD73-ND was determined. The SA protein was diluted to 1 μg/mL for coating the ELISA plates, and the biotinylated CD73 protein (CD73-biotin) was used at a concentration of 0.1 μg/mL. The experimental results are shown in FIGS. 18 to 21. The decline multiples of affinity (Ratio (EC₅₀)) and the decline multiples of high plateau value (Ratio (Top)) of each mutant protein for binding to 48A11-HuV33 are shown in Tables 9 to 12, respectively. As can be seen, the three amino acid sites Y132, L133, and P139 are the key sites affecting the binding of 48A11-HuV33 to CD73. After their mutations, the binding EC₅₀ decreases by more than 10 times, and the high plateau value Top decreases by more than 2 times. The next key sites are V137, L181, and L184, and after their mutations, the binding EC₅₀ decreases by 3-10 times or the high plateau value Top decreases by 1.5-2 times. Then there are sites V144 and K180, after their mutations, the binding EC₅₀ decreases by 2-3 times.

The positions of the above sites in the 3D crystal structure diagram of CD73 (sourced from PDB: 6VC9) are shown in FIG. 22, which further indicate that the binding epitope of 48A11-HuV33 to CD73 is a discontinuous spatial epitope, including the key amino acid sites mentioned above.

TABLE 9
Affinity of Each Mutant Protein for Binding to 48A11-HuV33

| Sample | EC₅₀(nM) | Ratio(EC₅₀) | Top | Ratio(Top) |
|---|---|---|---|---|
| CD73-ND | 0.152 | 1.0 | 2.084 | 1.0 |
| CD73-ND-Y132A | 10.520 | 69.2 | 0.254 | 8.2 |
| CD73-ND-L133A | 25.750 | 169.4 | 0.922 | 2.3 |
| CD73-ND-P134A | 0.205 | 1.3 | 1.974 | 1.1 |
| CD73-ND-Y135A | 0.165 | 1.1 | 1.964 | 1.1 |
| CD73-ND-K136A | 0.145 | 1.0 | 2.125 | 1.0 |
| CD73-ND-V137A | 0.891 | 5.9 | 1.601 | 1.3 |
| CD73-ND-L138A | 0.182 | 1.2 | 1.908 | 1.1 |

TABLE 10
Affinity of Each Mutant Protein for Binding to 48A11-HuV33

| Sample | EC₅₀(nM) | Ratio(EC₅₀) | Top | Ratio(Top) |
|---|---|---|---|---|
| CD73-ND | 0.117 | 1.0 | 1.982 | 1.0 |
| CD73-ND-E143A | 0.167 | 1.4 | 1.769 | 1.1 |
| CD73-ND-V144A | 0.290 | 2.5 | 1.685 | 1.2 |
| CD73-ND-K180A | 0.321 | 2.7 | 1.741 | 1.1 |
| CD73-ND-L181A | 0.226 | 1.9 | 1.051 | 1.9 |
| CD73-ND-K182A | 0.121 | 1.0 | 2.027 | 1.0 |
| CD73-ND-T183A | 0.159 | 1.4 | 1.975 | 1.0 |
| CD73-ND-N185A | 0.188 | 1.6 | 1.902 | 1.0 |

TABLE 11
Affinity of Each Mutant Protein for Binding to 48A11-HuV33

| Sample | EC₅₀(nM) | Ratio(EC₅₀) | Top | Ratio(Top) |
|---|---|---|---|---|
| CD73-ND | 0.178 | 1.0 | 1.998 | 1.0 |
| CD73-ND-P139A | 4.339 | 24.4 | 0.871 | 2.3 |
| CD73-ND-V140A | 0.098 | 0.6 | 2.053 | 1.0 |
| CD73-ND-D142A | 0.344 | 1.9 | 1.842 | 1.1 |
| CD73-ND-L184A | 0.612 | 3.4 | 1.703 | 1.2 |

TABLE 12
Affinity of Each Mutant Protein for Binding to 48A11-HuV33

| Sample | EC₅₀(nM) | Ratio(EC₅₀) | Top | Ratio(Top) |
|---|---|---|---|---|
| CD73-ND | 0.091 | 0.8 | 1.943 | 1.0 |
| CD73-ND-V186A | 0.160 | 1.4 | 1.618 | 1.2 |
| CD73-ND-N187A | 0.105 | 0.9 | 1.767 | 1.1 |

All literatures mentioned in the present application are incorporated herein by reference, as though each one is individually incorporated by reference. In addition, it should be understood that, after reading the above teachings of the present invention, those skilled in the art can make various changes or modifications to the present invention, these equivalents also falls in the scope as defined in the appended claims.

1. An anti-human CD73 antibody or an antigen-binding fragment thereof, which comprises a heavy chain variable region and a light chain variable region, wherein,
the heavy chain variable region comprises three heavy chain complementarity determining regions (CDRs):
HCDR1 represented by SEQ ID NO: 10,
HCDR2 represented by SEQ ID NO: 11,
HCDR3 represented by SEQ ID NO: 12; or
HCDR1 represented by SEQ ID NO: 16,
HCDR2 represented by SEQ ID NO: 17,
HCDR3 represented by SEQ ID NO: 18; and
the light chain variable region comprises three light chain complementarity determining regions (CDRs):
LCDR1 represented by SEQ ID NO: 13,
LCDR2 represented by SEQ ID NO: 14,
LCDR3 represented by SEQ ID NO: 15; or
LCDR1 represented by SEQ ID NO: 19,
LCDR2 represented by SEQ ID NO: 20,
LCDR3 represented by SEQ ID NO: 21;
wherein any one of the amino acid sequences of the antibody or the antigen-binding fragment thereof further comprises a derivative sequence that is optionally with at least one amino acid added, deleted, modified, and/or substituted, and is capable of retaining the affinity for binding to CD73.

2. The anti-human CD73 antibody or the antigen-binding fragment thereof of claim 1, wherein the heavy chain variable region has an amino acid sequence represented by SEQ ID NO: 1, 4, 6, 9, 24, or 28.

3. The anti-human CD73 antibody or the antigen-binding fragment thereof of claim 1, wherein the heavy chain constant region is of human origin or murine origin; preferably, the heavy chain constant region is a human antibody IgG1 or IgG4 constant region.

4. The anti-human CD73 antibody or the antigen-binding fragment thereof of claim 1, wherein the light chain variable region has an amino acid sequence represented by SEQ ID NO: 2, 3, 5, 7, 8, 26, or 30.

5. The anti-human CD73 antibody or the antigen-binding fragment thereof of claim 1, wherein the light chain constant region is of human origin or murine origin; preferably, the light chain constant region is a human antibody kappa chain constant region.

6. The anti-human CD73 antibody or the antigen-binding fragment thereof of claim 1, wherein the heavy chain variable region comprises any one of the amino acid sequences represented by SEQ ID NOs: 1, 4, 6, 9, 24, and 28; and/or the light chain variable region comprises any one of the amino acid sequences represented by SEQ ID NOs: 2, 3, 5, 7, 8, 26, and 30.

7. (canceled)

8. The anti-human CD73 antibody or the antigen-binding fragment thereof of claim 1, wherein the binding epitope of the antibody to human CD73 protein comprises a site corresponding to SEQ ID NO: 22 selected from the group consisting of:
tyrosine at position 132 (Y132), leucine at position 133 (L133), proline at position 139 (P139), valine at position 137 (V137), leucine at position 181 (L181), leucine at position 184 (L184), valine at position 144 (V144), and lysine at position 180 (K180).

9. A recombinant protein, which comprises:
(i) the antibody or the antigen-binding fragment thereof of claim 1; and
(ii) optionally a tag sequence for assisting expression and/or purification.

10. A polynucleotide encoding
the antibody or the antigen-binding fragment thereof of claim 1.

11. The polynucleotide of claim 10, wherein the polynucleotide encoding the heavy chain variable region is represented by SEQ ID NO: 33, 36, 38, 41, 23, or 27; and/or, the polynucleotide encoding the light chain variable region is represented by SEQ ID NO: 34, 35, 37, 39, 40, 25, or 29.

12. A vector comprising the polynucleotide of claim 10.

13. A genetically engineered host cell comprising the vector of claim 12.

14. An antibody conjugate, which comprises:
(a) an antibody moiety, which is the antibody or the antigen-binding fragment thereof of claim 1; and
(b) a conjugate moiety coupled to the antibody moiety, which is selected from the group consisting of: a detectable label, a drug, a toxin, a cytokine, a radionuclide, an enzyme, and a combination thereof.

15. A CAR construct, wherein the scFv segment of the monoclonal antibody antigen-binding region of the CAR construct is a binding region that specifically binds to CD73, and
wherein the heavy chain variable region of the scFv comprises the following three complementarity determining regions (CDRs):
HCDR1 represented by SEQ ID NO: 10,
HCDR2 represented by SEQ ID NO: 11,
HCDR3 represented by SEQ ID NO: 12; and
the light chain variable region of the scFv comprises the following three complementarity determining regions (CDRs):
LCDR1 represented by SEQ ID NO: 13,
LCDR2 represented by SEQ ID NO: 14,
LCDR3 represented by SEQ ID NO: 15; or
wherein the heavy chain variable region of the scFv comprises the following three complementarity determining regions (CDRs):
HCDR1 represented by SEQ ID NO: 16,
HCDR2 represented by SEQ ID NO: 17,
HCDR3 represented by SEQ ID NO: 18; and
the light chain variable region of the scFv comprises the following three complementarity determining regions (CDRs):
LCDR1 represented by SEQ ID NO: 19,
LCDR2 represented by SEQ ID NO: 20,
LCDR3 represented by SEQ ID NO: 21.

16. A recombinant immune cell, which expresses an exogenous CAR construct of claim 15.

17. A pharmaceutical composition, which comprises:
(i) an active ingredient, wherein the active ingredient is the antibody or the antigen-binding fragment thereof of claim 1; and
(ii) a pharmaceutically acceptable carrier.

18. (canceled)

19. A method for the preventing and/or treating a CD73-related disease, which comprises the step of: administering the antibody or the antigen-binding fragment thereof of claim 1, an antibody conjugate, a recombinant immune cell, or a pharmaceutical composition, and a combination thereof, to a subject in need thereof;
wherein the antibody conjugate comprises:
(a) an antibody moiety, which is the antibody or the antigen-binding fragment thereof of claim 1; and
(b) a conjugate moiety coupled to the antibody moiety, which is selected from the group consisting of: a detectable label, a drug, a toxin, a cytokine, a radionuclide, an enzyme, and a combination thereof;
wherein the recombinant immune cell expresses an exogenous CAR construct, wherein the scFv segment of the monoclonal antibody antigen-binding region of the CAR construct is a binding region that specifically binds to CD73, wherein the heavy chain variable region of the scFv comprises the following three complementarity determining regions (CDRs):
HCDR1 represented by SEQ ID NO: 10,
HCDR2 represented by SEQ ID NO: 11,
HCDR3 represented by SEQ ID NO: 12; and
the light chain variable region of the scFv comprises the following three complementarity determining regions (CDRs):
LCDR1 represented by SEQ ID NO: 13,
LCDR2 represented by SEQ ID NO: 14,
LCDR3 represented by SEQ ID NO: 15; or
wherein the heavy chain variable region of the scFv comprises the following three complementarity determining regions (CDRs):
HCDR1 represented by SEQ ID NO: 16,
HCDR2 represented by SEQ ID NO: 17,
HCDR3 represented by SEQ ID NO: 18; and
the light chain variable region of the scFv comprises the following three complementarity determining regions (CDRs):
LCDR1 represented by SEQ ID NO: 19,
LCDR2 represented by SEQ ID NO: 20,
LCDR3 represented by SEQ ID NO: 21; and
wherein the pharmaceutical composition comprises:
(i) an active ingredient, wherein the active ingredient is the antibody or the antigen-binding fragment thereof of claim 1; and
(ii) a pharmaceutically acceptable carrier.

20. The method of claim 19, wherein the CD73-related disease is selected from the group consisting of:
hematological cancer, lymphoma, glioblastoma, melanoma, skin cancer, gastric cancer, gastrointestinal stromal tumor, liver cancer, cholangiocarcinoma, gallbladder cancer, peritoneal cancer, colorectal cancer, small intestine cancer, anal cancer, multiple myeloma, pancreatic cancer, breast cancer, ovarian cancer, uterine cancer, cervical cancer, vaginal cancer, bladder cancer, renal cancer, non-small cell lung cancer, small cell lung cancer, prostate cancer, testicular cancer, penile cancer, thyroid cancer, head and neck cancer, esophageal cancer, bone cancer, and sarcoma.


SEQUENCE LISTING (41 sequences)

SEQ ID NO: 1 - protein, 119 aa, synthetic construct
  source 1..119: note = 56B10-HuV21 VH, 56B10-HuV23 VH
QVQLVQSGAEVKKPGASVKVSCKTSGYTFTRYYIYWVRQAPGQGLEWIGWIYPGNFNTKYNEKFKGRATLTADTSASTAYMELSSLRSEDTAVYYCARDVYDYAGFAYWGQGTLVTVSS

SEQ ID NO: 2 - protein, 107 aa, synthetic construct
  source 1..107: note = 56B10-HuV21 VL,56B10-HuV31 VL
DIQLTQSPSFLSASVGDRVTITCKASQGVATAVAWYQQKPGQSPKLLIYWASTRHTGVPDRFSGSGSGTEFTLTISSLQPEDFATYYCQQYSSYPWTFGQGTKVEIK

SEQ ID NO: 3 - protein, 107 aa, synthetic construct
  source 1..107: note = 56B10-HuV23 VL,56B10-HuV33 VL
DIQMTQSPSFLSASVGDRVTITCKASQGVATAVAWYQQKPGKSPKLLIYWASTRHTGVPSRFSGSGSGTEFTLTISSLQPEDFATYFCQQYSSYPWTFGQGTKVEIK

SEQ ID NO: 4 - protein, 119 aa, synthetic construct
  source 1..119: note = 56B10-HuV31 VH,56B10-HuV32 VH, 56B10-HuV33 VH
QVQLQQSGAEVKKPGASVKVSCKASGYTFTRYYIYWVRQRPGQGLEWIGWIYPGNFNTKYNEKFKGRATLTADTSASTAYMELSSLTSEDTAVYYCARDVYDYAGFAYWGQGTLVTVSS

SEQ ID NO: 5 - protein, 107 aa, synthetic construct
  source 1..107: note = 56B10-HuV33 VL
DIQLTQSPSFLSASVGDRVTITCKASQGVATAVAWYQQKPGKAPKLLIYWASTRHTGVPSRFSGSGSGTEFTLTISSLQPEDFATYYCQQYSSYPWTFGQGTKVEIK

SEQ ID NO: 6 - protein, 117 aa, synthetic construct
  source 1..117: note = 48A11-HuV22 VH,48A11-HuV23 VH
QVQLVQSGAEVKKPGASVKVSCKASGYTFTSYWMHWVRQAPGQGLEWIGEINPSIGRTNYNEKFKSRATLTVDKSTSTAYMELSSLRSEDTAVYYCARRVYGTMDYWGQGTLVTVSS

SEQ ID NO: 7 - protein, 106 aa, synthetic construct
  source 1..106: note = 48A11-HuV22 VL
DIQMTQSPSSLSASVGDRVTITCKASQDINSYLSWFQQKPGKSPKTLIYRANIWVDGVPSRFSGSGSGQDYTFTISSLQPEDIATYYCLQYDELYTFGQGTKVEIK

SEQ ID NO: 8 - protein, 106 aa, synthetic construct
  source 1..106: note = 48A11-HuV23 VL, 48A11-HuV33 VL
DIQMTQSPSSLSASVGDRVTITCKASQDINSYLSWFQQKPGKSPKLLIYRANIWVDGVPSRFSGSGSGQDYTFTISSLQPEDIATYYCLQYDELYTFGQGTKVEIK

SEQ ID NO: 9 - protein, 117 aa, synthetic construct
  source 1..117: note = 48A11-HuV33 VH
QVQLVQSGAEVKKPGASVKVSCKASGYTFTSYWMHWVRQAPGQGLEWIGEINPSIGRTNYNEKFKSRVTLTRDTSTSTAYMELSSLRSEDTAVYYCARRVYGTMDYWGQGTLVTVSS

SEQ ID NO: 10 - protein, 5 aa, synthetic construct
  source 1..5: note = 48A11 HCDR1
SYWMH

SEQ ID NO: 11 - protein, 17 aa, synthetic construct
  source 1..17: note = 48A11 HCDR2
EINPSIGRTNYNEKFKS

SEQ ID NO: 12 - protein, 8 aa, synthetic construct
  source 1..8: note = 48A11 HCDR3
RVYGTMDY

SEQ ID NO: 13 - protein, 11 aa, synthetic construct
  source 1..11: note = 48A11 LCDR1
KASQDINSYLS

SEQ ID NO: 14 - protein, 7 aa, synthetic construct
  source 1..7: note = 48A11 LCDR2
RANIWVD

SEQ ID NO: 15 - protein, 8 aa, synthetic construct
  source 1..8: note = 48A11 LCDR3
LQYDELYT

SEQ ID NO: 16 - protein, 5 aa, synthetic construct
  source 1..5: note = 56B10 HCDR1
RYYIY

SEQ ID NO: 17 - protein, 17 aa, synthetic construct
  source 1..17: note = 56B10 HCDR2
WIYPGNFNTKYNEKFKG

SEQ ID NO: 18 - protein, 10 aa, synthetic construct
  source 1..10: note = 56B10 HCDR3
DVYDYAGFAY

SEQ ID NO: 19 - protein, 11 aa, synthetic construct
  source 1..11: note = 56B10 LCDR1
KASQGVATAVA

SEQ ID NO: 20 - protein, 7 aa, synthetic construct
  source 1..7: note = 56B10 LCDR2
WASTRHT

SEQ ID NO: 21 - protein, 9 aa, synthetic construct
  source 1..9: note = 56B10 LCDR3
QQYSSYPWT

SEQ ID NO: 22 - protein, 523 aa, synthetic construct
  source 1..523: note = amino acid sequence of CD73 extracellular region
WELTILHTNDVHSRLEQTSEDSSKCVNASRCMGGVARLFTKVQQIRRAEPNVLLLDAGDQYQGTIWFTVYKGAEVAHFMNALRYDAMALGNHEFDNGVEGLIEPLLKEAKFPILSANIKAKGPLASQISGLYLPYKVLPVGDEVVGIVGYTSKETPFLSNPGTNLVFEDEITALQPEVDKLKTLNVNKIIALGHSGFEMDKLIAQKVRGVDVVVGGHSNTFLYTGNPPSKEVPAGKYPFIVTSDDGRKVPVVQAYAFGKYLGYLKIEFDERGNVISSHGNPILLNSSIPEDPSIKADINKWRIKLDNYSTQELGKTIVYLDGSSQSCRFRECNMGNLICDAMINNNLRHTDEMFWNHVSMCILNGGGIRSPIDERNNGTITWENLAAVLPFGGTFDLVQLKGSTLKKAFEHSVHRYGQSTGEFLQVGGIHVVYDLSRKPGDRVVKLDVLCTKCRVPSYDPLKMDEVYKVILPNFLANGGDGFQMIKDELLRHDSGDQDINVVSTYISKMKVIYPAVEGRIKFS

SEQ ID NO: 23 - DNA, 351 bp, synthetic construct
  source 1..351: mol_type = other DNA; note = nucleotide sequence of 48A11 murine heavy chain variable region
caggtccaactgcagcagcctggggctgaactggtgaagcctggggcttcagtgaagctgtcctgcaaggcttctggctacaccttcaccagctactggatgcactgggtgaagcagaggcctggacaaggccttgagtggattggagagattaatcctagcatcggtcgtactaactacaatgagaagttcaagagcaaggccacactgactgtagacaaatcctccagcacagccttcatgcaactcagcagtctgacatctgaggactctgcggtctattactgtgcaagaagggtctatggtactatggactactggggtcaaggaacctcagtcaccgtctcctca

SEQ ID NO: 24 - protein, 117 aa, synthetic construct
  source 1..117: note = amino acid sequence of 48A11 murine heavy chain variable region
QVQLQQPGAELVKPGASVKLSCKASGYTFTSYWMHWVKQRPGQGLEWIGEINPSIGRTNYNEKFKSKATLTVDKSSSTAFMQLSSLTSEDSAVYYCARRVYGTMDYWGQGTSVTVSS

SEQ ID NO: 25 - DNA, 318 bp, synthetic construct
  source 1..318: mol_type = other DNA; note = nucleotide sequence of 48A11 murine light chain variable region
gacatcaagatgacccagtctccatcttccatgtatgcatctctaggagagagagtcactatcacttgcaaggcgagtcaggacattaatagctatttaagctggttccagcagaaaccagggaaatctcctaagaccctgatctatcgtgcaaacatatgggtagatggggtcccatcaaggttcagtggcagtggatctgggcaagattattctctcaccatcagcagcctggagtatgaagatatgggaatttattattgtctacagtatgatgagttatacacgttcggaggggggaccaagctggaaataaaa

SEQ ID NO: 26 - protein, 106 aa, synthetic construct
  source 1..106: note = amino acid sequence of 48A11 murine light chain variable region
DIKMTQSPSSMYASLGERVTITCKASQDINSYLSWFQQKPGKSPKTLIYRANIWVDGVPSRFSGSGSGQDYSLTISSLEYEDMGIYYCLQYDELYTFGGGTKLEIK

SEQ ID NO: 27 - DNA, 357 bp, synthetic construct
  source 1..357: mol_type = other DNA; note = nucleotide sequence of 56B10 murine heavy chain variable region
caggtccagctgcagcagtctggacctgagctggtgaagcctggggcttcagtgaggatatcctgcaagacttctggctacaccttcacaagatactatatatattgggtgaagcagaggcctggacagggacttgagtggattggatggatttatcctggaaattttaatactaagtacaatgagaagttcaagggcaaggccacactgactgcagacacatcctccagcacagcctacatgcagctcagcagcctgacctctgaggactctgcggtctatttctgtgcaagagatgtatatgattacgcgggatttgcttactggggccaggggactctggtcactgtctctgca

SEQ ID NO: 28 - protein, 119 aa, synthetic construct
  source 1..119: note = amino acid sequence of 56B10 murine heavy chain variable region
QVQLQQSGPELVKPGASVRISCKTSGYTFTRYYIYWVKQRPGQGLEWIGWIYPGNFNTKYNEKFKGKATLTADTSSSTAYMQLSSLTSEDSAVYFCARDVYDYAGFAYWGQGTLVTVSA

SEQ ID NO: 29 - DNA, 321 bp, synthetic construct
  source 1..321: mol_type = other DNA; note = nucleotide sequence of 56B10 murine light chain variable region
gacattgtgatgacccagtctcacagattcttgtccacatcagtaggagacagggtcagcatcacctgcaaggccagtcagggtgtggctactgctgttgcctggtatcaacagaaaccaggacaatctcctaaactcctgatttactgggcatccacccggcacactggagtccctgatcgcttcacaggcagtggatctgggacagatttcactctcaccattagcaatgtgcagtctgaagacttggcagattatttctgtcagcaatatagcagctatccgtggacgttcggtggaggcaccaagctggaaatcaaa

SEQ ID NO: 30 - protein, 107 aa, synthetic construct
  source 1..107: note = amino acid sequence of 56B10 murine light chain variable region
DIVMTQSHRFLSTSVGDRVSITCKASQGVATAVAWYQQKPGQSPKLLIYWASTRHTGVPDRFTGSGSGTDFTLTISNVQSEDLADYFCQQYSSYPWTFGGGTKLEIK

SEQ ID NO: 31 - protein, 327 aa, synthetic construct
  source 1..327: note = amino acid sequence of human IgG4 (S228P) constant region
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 32 - protein, 107 aa, synthetic construct [sequence omitted]

SEQ ID NO: 33 - DNA, 357 bp, synthetic construct
  source 1..357: mol_type = other DNA; note = 56B10-HuV21 VH, 56B10-HuV23 VH
caggtgcagctggtgcagtctggcgctgaagtgaagaagcctggcgcctctgtgaaggtgagctgcaagacctccggctacaccttcaccaggtactacatctactgggtgaggcaggcccccggccagggcctggagtggatcggctggatctaccccggcaactttaacaccaagtacaacgagaagttcaagggccgggccaccctgaccgccgacacctctgccagcaccgcctacatggagctgtctagcctgcggtctgaggacaccgccgtgtactactgcgctcgggacgtgtacgactacgccggcttcgcctactggggccagggcaccctggtgaccgtgagctct

SEQ ID NO: 34 - DNA, 321 bp, synthetic construct
  source 1..321: mol_type = other DNA; note = 56B10-HuV21 VL,56B10-HuV31 VL
gacatccagttaacccagtccccctcctttctgagcgcctccgtgggagacagggtcaccatcacctgtaaggcctctcagggggtggctacagcagtagcctggtatcaacagaagcctggccagtctcctaagctgctgatatattgggcgtctactcggcacactggggtgcccgacaggttcagcggctccggttccggcaccgagttcaccctgaccatctcctccctgcagcccgaggacttcgccacgtactattgccaacagtactcctcctacccctggaccttcggccagggcaccaaagtggagatcaag

SEQ ID NO: 35 - DNA, 321 bp, synthetic construct
  source 1..321: mol_type = other DNA; note = 56B10-HuV23 VL,56B10-HuV33 VL
gatatccagatgacccagtctccatctttcctgtctgctagcgtgggagatagagtgacaattacttgtaaggctagccagggagtggctaccgccgtggcttggtaccagcagaagcctggcaagtctcctaagctgctgatctattgggcttccactagacataccggagtgccttccagattttctggatctggctccggaactgagtttacactgacaatctcttctctgcagcctgaggattttgccacatatttttgtcagcagtattcttcttatccatggacatttggccagggcacaaaggtggagattaag

SEQ ID NO: 36 - DNA, 357 bp, synthetic construct
  source 1..357: mol_type = other DNA; note = 56B10-HuV31 VH,56B10-HuV32 VH, 56B10-HuV33 VH
caggtgcagctgcaacagtctggcgctgaagtgaagaagcctggcgcctctgtgaaggtgagctgcaaggcatccggctacaccttcaccaggtactacatctactgggtgaggcagagacccggccagggcctggagtggatcggctggatctaccccggcaactttaacaccaagtacaacgagaagttcaagggccgggccaccctgaccgccgacacctctgccagcaccgcctacatggagctgtctagcctgacatctgaggacaccgccgtgtactactgcgctcgggacgtgtacgactacgccggcttcgcctactggggccagggcaccctggtgaccgtgagctct

SEQ ID NO: 37 - DNA, 321 bp, synthetic construct
  source 1..321: mol_type = other DNA; note = 56B10-HuV33 VL
gacatccagttaacccagtccccctcctttctgagcgcctccgtgggagacagggtcaccatcacctgtaaggcctctcagggggtggctacagcagtagcctggtatcaacagaaacctggcaaagctcccaagctgctgatatattgggcgtctactcggcacactggggtgccctccaggttcagcggctccggttccggcaccgagttcaccctgaccatctcctccctgcagcccgaggacttcgccacgtactattgccaacagtactcctcctacccctggaccttcggccagggcaccaaagtggagatcaag

SEQ ID NO: 38 - DNA, 351 bp, synthetic construct
  source 1..351: mol_type = other DNA; note = 48A11-HuV22 VH,48A11-HuV23 VH
caggtgcagctggtgcagtccggcgccgaggtgaagaagccaggcgcctccgtgaaggtgagctgcaaggcctccggctacaccttcacctcctactggatgcactgggtgcggcaggcccctggccagggcctggagtggatcggcgagatcaaccccagcatcggccgcaccaactataacgagaagttcaagagccgggccaccctgaccgtggataagtccacctccaccgcctacatggagctgagctccctgaggtccgaggacaccgccgtgtactactgcgcccggcgcgtgtacggcaccatggactactggggccagggcaccctggtgaccgtgagcagc

SEQ ID NO: 39 - DNA, 318 bp, synthetic construct
  source 1..318: mol_type = other DNA; note = 48A11-HuV22 VL
gacatccagatgacccagtcccccagctccctgagcgcctccgtgggcgacagggtgaccatcacctgcaaggcctcccaggacatcaactcctacctgtcctggttccagcagaagcccggcaagtcccctaagaccctgatctacagggccaacatctgggtggacggcgtgccctccaggttcagcggctccggcagcggccaggactacaccttcaccatctcctccctgcagcccgaggacatcgccacctactactgcctgcagtacgacgagctgtacaccttcggccagggcaccaaggtggagatcaag

SEQ ID NO: 40 - DNA, 318 bp, synthetic construct
  source 1..318: mol_type = other DNA; note = 48A11-HuV23 VL, 48A11-HuV33 VL
gacatccagatgacccagtcccccagctccctgagcgcctccgtgggcgacagggtgaccatcacctgcaaggcctcccaggacatcaactcctacctgtcctggttccagcagaagcccggcaagtcccctaagctactgatctacagggccaacatctgggtggacggcgtgccctccaggttcagcggctccggcagcggccaggactacaccttcaccatctcctccctgcagcccgaggacatcgccacctactactgcctgcagtacgacgagctgtacaccttcggccagggcaccaaggtggagatcaag

SEQ ID NO: 41 - DNA, 351 bp, synthetic construct
  source 1..351: mol_type = other DNA; note = 48A11-HuV33 VH
caggtgcagctggtgcagtccggcgccgaggtgaagaagcccggcgccagcgtgaaggtgtcctgcaaggccagcggctacaccttcacctcctactggatgcactgggtgaggcaggcccccggccagggcctggagtggattggcgagatcaaccccagcatcggcaggaccaactacaacgagaagttcaagagcagggtgaccttgaccagagacacgtccaccagcaccgcttacatggagctgagctctctgaggtctgaggacaccgccgtgtactactgcgccaggcgggtgtacggcaccatggactactggggccagggcaccctggtgaccgtgagctcc